(19) United States
(12) Patent Application Publication
(10) Pub. No.: US 2010/0056648 A1
(43) Pub. Date: Mar. 4, 2010

(54) Processes and systems for production of liquefied petroleum gas (LPG)

(76) Inventors: Joseph W. Schroer, Chino Hills, CA (US); Drow Lionel O'Young, Walnut, CA (US); Vaibhav Kelkar, Chino Hills, CA (US); Kaoru Fujimoto, Kitakyushu City (JP); Hiroshi Kaneko, Yokohama City (JP)

Correspondence Address: MORGAN, LEWIS & BOCKIUS, LLP. (PA), 2 PALO ALTO SQUARE, 3000 EL CAMINO REAL, SUITE 700, PALO ALTO, CA, 94306, US

(21) Appl. No.: 12/460,878

(22) Filed: Jul. 24, 2009

Related U.S. Application Data
(60) Provisional application No. 61/083,397, filed on Jul. 24, 2008.

(51) Int. Cl. C07C 27/00; B01J 8/00

(52) U.S. Cl. 518/703; 518/705; 422/187

(57) ABSTRACT

The present invention relates generally to processes and systems for the production of liquefied petroleum gas (LPG). More specifically, embodiments of the present invention relate to improved methods and systems for the direct reaction of synthesis gas (syngas) to liquefied petroleum gas.

### CROSS REFERENCE TO RELATED APPLICATIONS

This application claims the benefit of, and priority to, U.S. Provisional Patent Application Ser. No. 61/083,397 filed on Jul. 24, 2008 entitled “Processes and Systems for Production of Liquefied Petroleum Gas (LPG),” the entire disclosure of which is incorporated by reference herein.

### FIELD OF THE INVENTION

The present invention relates generally to processes and systems for the production of liquefied petroleum gas (LPG). More specifically, embodiments of the present invention relate to improved methods and systems for the direct reaction of synthesis gas (syngas) to liquefied petroleum gas.

### BACKGROUND OF THE INVENTION

With the continued increasing energy demand by developing and developed countries, providing new sources of energy, particularly fuels, has become of paramount importance. Interest in natural gas has taken on added significance, particularly in connection with the conversion of natural gas into other, highly valued products.

Historically, processes to convert natural gas into hydrocarbons have been developed; the key process being by Fischer-Tropsch (F-T) synthesis. In general, the F-T process converts synthesis gas (syngas), a mixture of carbon monoxide and hydrogen, into liquid hydrocarbons with the aid of catalysts. Traditional catalysts include iron and cobalt based catalysts.

More recently, significant attention has been focused on liquefied petroleum gas (LPG). In general, LPG is comprised mainly of propane or butane, and may be readily stored and transported.

Developments in the production of LPG have been made. Established technologies currently available include: methanol synthesis from syngas, DME synthesis from methanol, olefins synthesis from methanol and/or DME, and olefins hydrogenation to LPG components. While these developments have been made, such processes require multiple reactors to carry out multiple reactions, separators, and the like, all with their attendant energy requirements. Thus, further innovation is needed.

Most recently, new catalysts have been developed under the direction of Professor Kaoru Fujimoto at the University of Kitakyushu Department of Chemical Processes and Environments. As described in detail in U.S. Pat. No. 7,297,825, a catalyst comprised of a Pd-based methanol synthesis catalyst component and a β-zeolite catalyst component has been advantageously employed in the production of LPG. This new catalyst enables more direct conversion of syngas to LPG.

By combining all these reactions, an LPG production process at a much lower cost can potentially be developed. A one-step or so-called “Direct” process from syngas to LPG would be advantageous for the production of LPG on-demand. Since fewer reactors are needed, the simplicity of the process should make it cost effective compared to other chemical production routes for making LPG. Accordingly, further developments are highly desired.

### SUMMARY OF THE INVENTION

After substantial study and research the inventors have discovered improved processes and systems for the direct reaction of syngas to liquefied petroleum gas (LPG), sometimes referred to as “Direct LPG” reaction or conversion. In summary, embodiments of the present invention provide methods and systems for production of liquefied petroleum natural gas. In summary, certain of the key innovations in the new process can be categorized in the following areas: Overall system configuration, the Reformer configuration, and the Separation system, among others.

In some embodiments a method for producing liquefied petroleum gas (LPG), is provided comprising the steps of: reacting carbon monoxide and hydrogen in the presence of a catalyst in an LPG reactor, wherein carbon dioxide is recycled to a reformer unit and produces a feed stream to the LPG reactor, and wherein the ratio of H₂ to CO in the LPG reactor is greater than the ratio of H₂ to CO in the reformer.

In some embodiments a method of producing liquefied petroleum gas (LPG) is provided characterized in that a reformer unit is operated with a feed stream of carbon dioxide and oxygen and without steam. Thus, in some embodiments the process is carried out without steam reforming, contrary to prior art processes.

In another aspect of the present invention a method for producing liquefied petroleum gas (LPG), comprising the steps of: producing synthesis gas in a reformer unit from a carbon containing material and oxygen; and reacting the synthesis gas in the presence of a catalyst in an LPG reactor, wherein a ratio of H₂ to CO in the LPG reactor is greater than the ratio of H₂ to CO in the reformer.

In some embodiments the ratio of H₂ to CO in the LPG reactor is in the range of up to approximately 2.0. In some embodiments the reaction is carried out at a pressure of 2.2 MPa or lower, alternatively at a pressure of 6 MPa or lower. In some embodiments the reaction is carried out at a temperature of 320° C. or lower, alternatively in the range of 260° C. to 360° C.

In some embodiments the reforming step wherein synthesis gas is produced is carried out without steam. In some embodiments separating LPG is carried out in a cryogenic separation system.

Of one advantage, embodiments of the present invention provide for selectively controlling the H₂ to CO ratio. In one embodiment this is achieved by recycling carbon oxides to the reformer unit to selectively control the H₂ to CO ratio. In another embodiment this is achieved by recycling hydrogen to the feed stream of the LPG reactor to selectively control the H₂ to CO ratio.

In another aspect of embodiments of the present invention, a system for producing liquefied petroleum gas (LPG), comprising: a reformer unit having a first ratio of H₂ to CO; an LPG reactor having a second H₂ to CO; and a separator system, wherein the second ratio of H₂ to CO is greater than the first ratio of H₂ to CO. In some embodiments the separator system is comprised of a cryogenic separations system. In some embodiments the reformer unit is configured to operate without steam.

### BRIEF DESCRIPTION OF THE FIGURES

The skilled artisan will understand that the drawings, described below, are for illustration purposes only. The drawings are not intended to limit the scope of the present teachings in any way.

FIG. 1 is a flowsheet showing a LPG system according to one embodiment of the present invention;

FIG. 2 illustrates Component flows vs. catalyst mass for kinetic model, T=350° C., P=50.333 atm, and argon carrier gas is 3.288 kmol/h;

FIG. 3 shows Component flows vs. catalyst mass for kinetic model, T=350° C., P=50.333 atm, and argon carrier gas is 3.288 kmol/h (zoomed in y-axis);

FIG. 4 shows Component flows vs. catalyst mass for kinetic model, T=400° C., P=50.333 atm, and argon carrier gas is 13.511 kmol/h;

FIG. 5 depicts Component flows vs. catalyst mass for kinetic model, T=400° C., P=50.333 arm, and argon carrier gas is 13.511 kmol/h (zoomed in y-axis);

FIG. 6 shows a Comparison of experimental data and kinetic model for CO conversion vs. WIF;

FIG. 7 shows a Comparison of experimental data and kinetic model for CO conversion vs. pressure;

FIG. 8 shows a Comparison of experimental data and kinetic model for CO conversion vs. temperature;

FIG. 9 shows a Comparison of experimental data and model for hydrocarbon distribution vs. pressure;

FIG. 10 shows a Comparison of experimental data and model for hydrocarbon distribution vs. temperature;

FIG. 11 illustrates a Model prediction of effect of CO₂ in syngas;

FIG. 12 shows a Model prediction of CO₂ concentration at reaction equilibrium;

FIG. 13 illustrates a Model prediction of the effect of methane and nitrogen on the LPG reaction;

FIG. 14 depicts Experimental data of new catalyst and kinetic model for CO conversion vs. W/F;

FIG. 15 shows Experimental data of new catalyst and kinetic model for CO conversion vs. pressure;

FIG. 16 shows a Comparison of experimental data of new catalyst and kinetic model for hydrocarbon distribution vs. W/F;

FIG. 17 shows a Comparison of experimental data of new catalyst and kinetic model for hydrocarbon distribution vs. temperature;

FIG. 18 shows a Comparison of experimental data of new catalyst and kinetic model for hydrocarbon distribution vs. pressure;

FIG. 19 illustrates Hydrocarbon yield versus H₂:CO;

FIG. 20 shows the Ratio of carbon in formed CO₂ to carbon in formed hydrocarbons versus H₂:CO;

FIG. 21A is a process schematic of a one-pass reactor material balance for one process design embodiment of the present invention;

FIG. 21B is a process schematic of a one-pass reactor material balance according to one embodiment of the present invention;

FIG. 22 is a process schematic showing a system according to some embodiments of the present invention with one reactor and one separation system;

FIG. 23 is a process schematic of a methanation system according to some embodiments of the present invention;

FIG. 24 is a simplified process schematic of one embodiment of a superstructure process according to the present invention;

FIG. 25 is a process schematic showing a system according to some embodiments of the present invention;

FIG. 26 is a process schematic showing a system according to other embodiments of the present invention;

FIG. 27 is a process schematic showing a system according to yet other embodiments of the present invention;

FIG. 28 is a process schematic showing a system according to another embodiment of the present invention which combines the systems of G and H;

FIGS. 29A-29C illustrate Compressor and expander placements for various LPG reaction pressures. (a) P(direct)>=2 MPa>P(sep)>P(reformer), (b) P(sep)>P(reformer)>1 MPa <=P(direct), (c) P(sep)>P(reformer)˜1-2 MPa˜P(direct);

FIG. 30 illustrates a material balance assuming partial oxidation reforming and no recycle;

FIG. 31 illustrates natural gas to LPG carbon efficiency for H₂:CO=0.67;

FIG. 32 illustrates natural gas to LPG carbon efficiency for H₂:CO=1.0;

FIG. 33 illustrates natural gas to LPG carbon efficiency for H₂:CO=2.0;

FIG. 34 illustrates natural gas to LPG carbon efficiency for H₂:CO=2.8;

FIG. 35 shows a detailed schematic of one embodiment of a separation scheme;

FIG. 36 shows a detailed schematic of another embodiment of a separation scheme;

FIG. 37 shows a detailed schematic of a three column embodiment of a separation scheme;

FIG. 38 shows an absorption recovery embodiment system according to some embodiments;

FIG. 39 illustrates a simulation embodiment for heavy components absorption;

FIG. 40 illustrates another simulation embodiment for heavy components absorption; and

FIG. 41 illustrates a schematic showing one water quench embodiment.

### DETAILED DESCRIPTION OF THE INVENTION

The present invention provides systems and methods of producing liquefied petroleum natural gas (LPG). To facilitate understanding of the invention, this description is divided into sections below.

A. Overview

1. Overall System Configuration

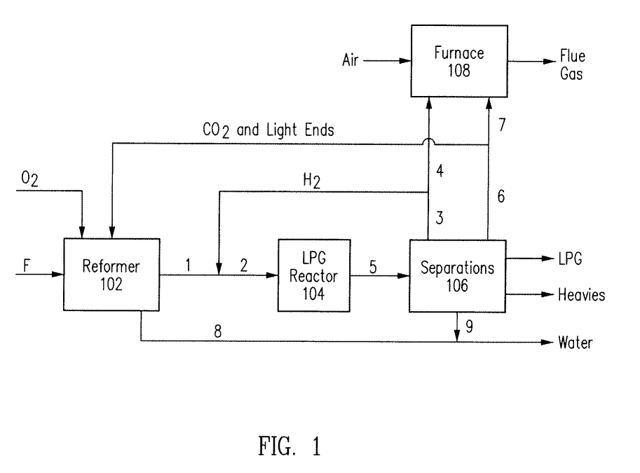

One embodiment of the system of the present invention is illustrated in FIG. 1. In general, the system 100 is comprised of a reformer 102, LPG reactor 104 and separation system or unit 106. A furnace 108 may also be provided. In general, the process is carried out wherein a carbon containing source, such as hydrocarbon fuel or natural gas F, and oxygen are fed to reformer 102 to produce primarily CO and CO₂ (in output stream 1). Of particular development, reformer 102 is operated without steam. Output stream 1 is then selectively mixed with recycled hydrogen to produce feed stream 2 of a desired hydrogen to carbon monoxide ratio which is then fed to LPG reactor 104. The carbon monoxide and hydrogen in feed stream 2 are reacted in LPG reactor 104 in the presence of a catalyst to produce output stream 5 which contains LPG.

Output stream 5 is then sent to separation system 106 where the LPG is separated from stream 5 to produce the LPG product comprising a mixture of primarily propane and butane. Carbon monoxide, carbon dioxide and light ends are separated from stream 5 and recycled to the reformer 102 via stream 6. Hydrogen is separated from stream 5 and recycles to the output of the reformer via stream 3. Some light components are sent to furnace 108 and exit as flue gas. Water and the bottom components (also referred to as “heavies”) are separated from the bottom of the separation system 106.

In some embodiments, one key feature is that some carbon dioxide, as well as hydrocarbons lighter than LPG components (sometimes referred to as “light end”), are recycled to the reformer 102. Hydrogen is not recycled to the reformer but recovered and mixed with the feed stream 2 to the LPG reactor 104 to control the hydrogen to carbon monoxide (H₂:CO) ratio. In general, the ratio of H₂ to CO in the LPG reactor 104 is higher than the outlet stream 1 of the reformer 104. In one embodiment the ratio of H₂ to CO in the feed stream 2 to the LPG reactor 104 is about 2.0. This ratio provides improved LPG reactor performance and was unexpected. In fact, this ratio can be higher than the actual product ratio.

Referring again to FIG. 1, one example of an exemplary overall material balance for a plant with 400 kTA capacity and being stand-alone type is given in Tables 1A and 1B below:

TABLE 1A
Exemplary overall material balance for the Direct Process.
Material flows in T/H. Refer to FIG. 1 for stream location.

| | Stream | | | | | | | | |
|---|---|---|---|---|---|---|---|---|---|
| | 1 | 2 | 3 | 4 | 5 | 6 | 7 | 8 | 9 |
| H₂ | 25.3 | 46.2 | 23.4 | 2.5 | 25.1 | 1.7 | 0.5 | | |
| CO | 320.9 | 320.9 | | | 34.3 | 34.3 | 10.3 | | |
| N₂, Ar | 35.6 | 35.6 | | | 35.6 | 35.6 | 10.7 | | |
| C1-C2 | 2.5 | 2.5 | | | 23.7 | 23.5 | 7.0 | | |
| CO₂ | 86.8 | 86.8 | | | 268.7 | 268.6 | 80.6 | 0.1 | 0.2 |
| C3-C4 | | | | | 50.2 | 0.6 | 0.2 | | |
| C5⁺ | | | | | 19.0 | | | | |
| H₂O | 1.9 | 1.9 | | | 37.3 | | | 72.8 | 37.3 |
| O₂ | | | | | | | | | |

TABLE 1B
Exemplary overall material balance for the Direct Process. Material flows in T/H.
Refer to FIG. 1 for stream location

| | | | CO₂ | | | | | | |
|---|---|---|---|---|---|---|---|---|---|
| | | | and Light | | | | | | |
| Stream | F | O₂ | Ends | H₂ | LPG | Heavies | Water | Air | Flue Gas |
| H₂ | | | 1.2 | 20.9 | | | | | |
| CO | | | 24.0 | | | | | | |
| N₂, Ar | | 10.7 | 24.9 | | | | | 229.5 | 240.1 |
| C1-C2 | 117.9 | | 16.4 | | 0.2 | | | | |
| CO₂ | | | 188.0 | | | | 0.3 | | 116.9 |
| C3-C4 | | | 0.4 | | 49.6 | | | | |
| C5⁺ | | | | | 0.2 | 18.8 | | | |
| H₂O | | | | | | | 110.1 | | 42.2 |
| O₂ | | 162.4 | | | | | | 69.7 | 11.6 |

2. Reformer

In another aspect of the present invention, a new reformer system and method are provided. Of particular advantage, the reformer 102 is configured such that the process does not use steam, and instead uses recycled carbon dioxide combined with oxygen. This utilizes effectively the byproduct carbon dioxide generated in the LPG reaction as well as maintains conversion in the reformer and generates a suitable H₂:CO ratio for the LPG product while minimizing excess hydrogen generation. Steam reforming in contrast will generate excess hydrogen that is not desirable for the process of the present invention.

3. Separation System

The separation system 106 is described in more detail below. In some embodiments, separation system 106 is configured for cryogenic recovery of LPG. In an exemplary embodiment, carbon dioxide and water are first removed in the separation system to avoid solidification in the cryogenic LPG. This also allows for CO₂ recycle to the reformer 102. CO₂ handling is an advantageous aspect of the overall system according to embodiments of the present invention. Additionally, embodiments of the separation system of the present invention are configured to remove water before LPG recovery to avoid solidification in the cryogenic LPG recovery system. This also avoids formation of a water-butane azeotrope.

Moreover, according to some embodiments of the present invention it is preferred that hydrogen be separated are recycled to selectively control the H₂:CO ratio of the feed stream fed to the LPG reactor. Removal of hydrogen before LPG recovery further reduces the gas volume to the LPG recovery section of the separation system and raises the LPG partial pressure so the capital cost of the LPG recovery section is reduced. Managing the H₂:CO ratio at the various parts of the process is an advantageous aspect according to embodiments of the overall system of the present invention.

4. Summary of One Exemplary Embodiment

In one exemplary embodiment, the method is carried out at a pressure in the range of up to 2.2 MPa. Catalyst modifications have allowed higher conversion at lower pressure. In another embodiment, the pressure is below about 2.2 MPa, and in an alternative embodiment, the pressure is less than 6 MPa. A mix of powder/finely ground catalysts may be employed. In one example, catalysts are employed as described in U.S. Pat. No. 7,297,825, the entire disclosure of which is hereby incorporated by reference. In the exemplary embodiment, the method is carried out at a temperature of 320° C. or lower, preferably to keep the selectivity high. In another embodiment, the temperature range is between 260° C. and 360° C., but preferably below 320° C. The equilibrium concentration of CO₂ is relatively high, around 40%, making the volume of CO₂ recycled high. The heat of reaction is large and in fact is a major consideration in the reactor design. For 80% conversion, the adiabatic temperature rise for the reactor would be 800° C. Feeding cool gas can only reduce a small portion of the heat. The reactor design preferably includes a heat removal method. The present invention is specifically suitable for converting natural gas to LPG. Of particular advantage, the reformer employs recycled carbon dioxide as well as fresh oxygen feed as the oxygen source. In some embodiments, the method employs cooling followed by vapor-liquid separation, which preferably uses fewer distillation columns and lowers capital cost.

B. LPG Reactor Modeling

To illustrate some embodiments of the present invention, a number of reactor models where developed; more specifically, modeling the reactor performance of chemical reaction of the methods and systems herein. The next section briefly describes the properties of the models used.

1. Data Point Model (Fixed Selectivity and Conversion)

This model assumes that the output of the chemical reactor has a fixed conversion of reactants and fixed selectivity. The selectivity and conversion is specified as the selectivity and conversion determined from the laboratory experiment. In an alternate mode, sensitivity analysis can be performed by calculating the process performance by assuming various values for the conversion and selectivity for reactants and products.

For design purposes, this is the simplest model, and, if based on experimental data can be very good for a reasonable estimation of many chemical processes. However it has a drawback that is particularly of concern in the LPG from syngas case. By using this model, we are not able to study the effect of changes in the syngas feed composition except by linking to experimental data that also varies the feed composition. In this process there are many possibilities for the actual syngas composition. For example the hydrogen to carbon monoxide ratio can be varied as we change the oxidant feed rate or type of reformer producing the syngas. The carbon dioxide content of the syngas can also be changed by reformer conditions or by a water-gas shift converter. Inert gases such as nitrogen or argon can accumulate in the system, thus lowering the partial pressure of reactants. Unconverted methane from the natural gas may also be contained in the syngas feeding the LPG reactor. Hydrogen and other components can be separated from the tail gas and then remixed with the feed to the LPG reactor, also changing the feed composition. The LPG reactor will have different performance depending on the hydrogen to carbon monoxide feed ratio.

2. Equilibrium Reaction Model

The equilibrium reaction model calculates the outlet of the reactor based on the inlet conditions and specified reactor conditions by the minimization of the Gibbs free energy of the mixture components. This can be an unconstrained calculation, assuming all reactants and products can interconvert, or a constrained calculation, assuming only certain reactions or a certain temperature approach to the equilibrium takes place. An equilibrium reaction model was assumed for predicting reformer performance.

Equilibrium reactor performance is also valid for many chemical reactions. However it may not be valid under certain conditions. If the reaction products do not reach their full equilibrium concentration in the reactor then the reaction is kinetically controlled and a kinetic model is needed instead. If the catalyst is a shape-selective catalyst or the desired product is not thermodynamically favored compared to the byproducts then there should be cause for using the kinetic model.

3. Kinetic Reaction Model

The kinetic reaction model assumes the reaction proceeds according to the law of mass action and that the conversion of reactants to products varies with the amount of catalyst in the reactor. This model relates the rate of chemical reaction to the concentration of chemical species, catalyst concentration, and external conditions such as pressure and temperature at any point in the reactor. This forms a set of ordinary differential equations that are solved to predict the reactor output. This model can be combined with the reactor flow model etc. for the purposes of reactor design calculations, etc.

There are two types of kinetic models, microkinetic and macrokinetic models. Microkinetic models represent the detailed and actual reaction steps that are believed to take place on the catalyst and reflect our actual understanding of the reaction mechanism. Since research is ongoing for the direct LPG synthesis reaction the actual mechanism is still under debate by scientists. Therefore the kinetic model that is employed in this project is a macrokinetic model. This model is regressed to be able to predict the material balance of the reactor in terms of only the main chemical components. The equations may differ somewhat from the actual chemical mechanism, but should represent the material balances of the components in the reactor.

Kinetic models are capable of accurately representing the output of the reaction with changes to the input feed composition, provided they are based on data representing the range of composition of interest. Extrapolation of the model beyond the data it has been regressed from can give incorrect results. In the LPG synthesis reaction there are many components and many reactions taking place. Therefore kinetic models for this reaction should be evaluated carefully and only used within the range of parameters it is shown to accurately represent the results.

4. Combined Model

Kinetic models, equilibrium models, point models, and other reaction models can be combined with each other and with other unit operations to give a conceptual model of the reaction. For the kinetic model implementation in the simulator, we actually use a combined model that also calculates the n-butane-isobutane equilibrium, a detail that affects the separation system performance but was not included in the kinetic model.

C. Reaction Description

The overall reaction can be described by the following equation (Zhang et al., 2005):

2nCO+(n+1)H₂→CₙH₂ₙ₊₂+nCO₂

with the heat of reaction being approximately −50 n kcal/mol. The catalyst used for the reaction is a physical mixture of methanol synthesis catalyst and proton-type zeolite catalyst. Although the formulation under research was partially published in the literature, the exact formulation of the current catalyst used was not made known to CWB. Two catalysts are used to promote two functions. Firstly is the synthesis of methanol from the syngas. Secondly is the dehydration of methanol to dimethyl ether (DME) and hydrocarbon synthesis to LPG. Zeolite catalyst is commonly used for the dehydration of alcohols. The mechanism of the reaction is believed to proceed in this way. First, methanol is formed from the syngas. This is believed to be the limiting reaction step. After methanol is formed it is converted to DME. The DME further reacts to form hydrocarbons. It is also generally believed that the water-gas-shift reaction may occur on the catalyst. Alkane cracking reactions may also take place, even at temperatures as low as 240° C.

The reaction is fairly selective to LPG production. The formation of unsaturated hydrocarbons and aromatics is considered to be negligible.

Analysis of the reaction data yielded some interesting results. Initially it was thought that simply competition between the hydrogenation catalyst and the hydrocarbon polymerization catalyst would dictate the results. The hydrocarbons were also assumed to grow by chain growth. Under that assumption with high hydrogenation catalyst loading one would expect methane and ethane be the dominant products.

However it was found that high concentrations of higher hydrocarbon chains are present even with a lot of hydrogenation catalyst present. Thus equations to represent aggregate hydrocarbon growth to the kinetic model were added. It was also noted that the hydrocarbon distribution did not change much with hydrogen concentration, which also supports the aggregate growth hypothesis.

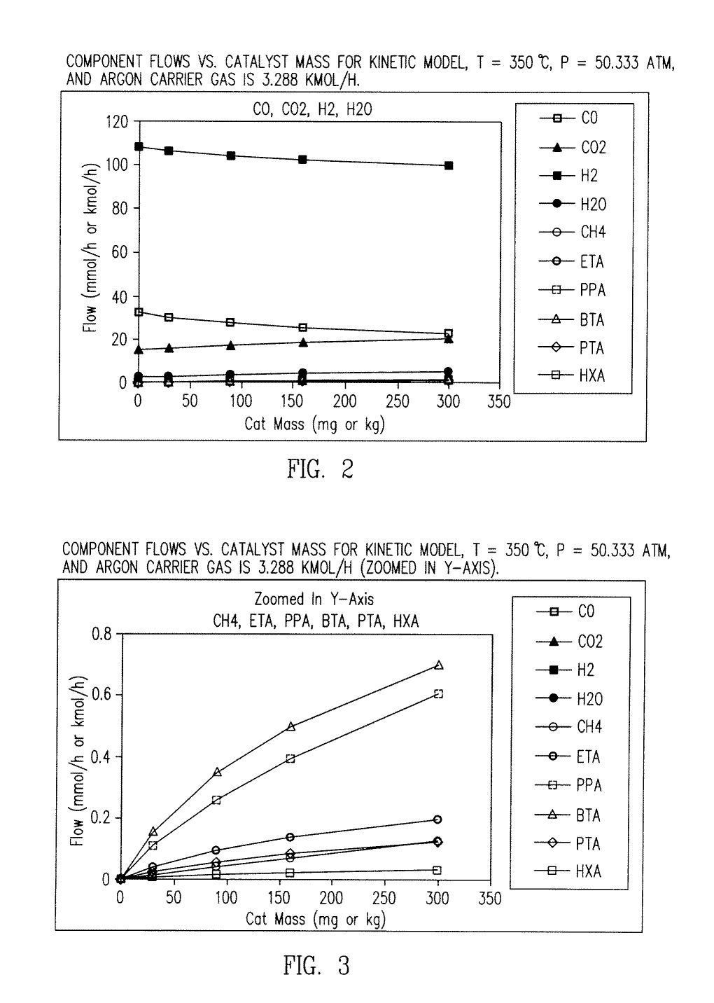

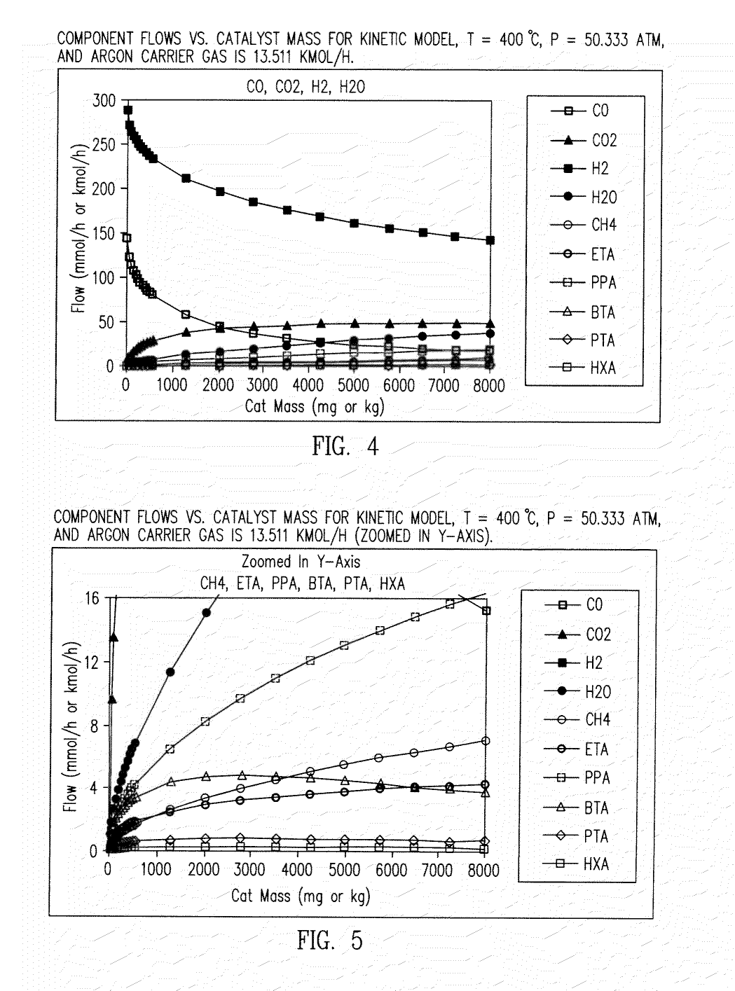

FIG. 2 to FIG. 5 plot calculations implemented in Aspen plus software of methods of the present invention on a side by side basis. FIG. 2 and FIG. 3 are for a run where the case is T=350° C., P=50.333 atm, and argon carrier gas is 3.288 kmol/h. FIG. 4 and FIG. 5 are for a run where the case is T=400° C., P=50.333 atm, and argon carrier gas is 13.511 kmol/h. Although there are some minor technical differences in the calculations performed by using REX verses Aspen, the agreement in the numerical values of the calculation results is excellent, showing the model has been successfully transferred to the process simulator.

The set of reactions selected for the kinetic model is given in Table 2. As can be seen there are 10 reactions. The model is not a mechanistic model, as seen by the absence of methanol and DME formation reactions, but a simplified model designed to correlate the material balance of the reactor. It does include the water-gas shift reaction. The assumption of no methanol or DME in the outlet is reasonable because it is rapidly converted to hydrocarbons. The experimentally measured concentration of methanol and DME was low. Also we suppose that the reactor design should minimize the concentration in the outlet to avoid unduly burdening the separation system.

TABLE 2
Stoichiometric equations of the kinetic model

| ID | Stoichiometric Equation |
|---|---|
| R_CO2: | CO + H₂O [symbol] H₂ + CO₂ (At equilibrium) |
| R_CH4: | 3H₂ + CO → CH₄ + H₂O |
| R_ETA: | 5H₂ + 2CO → ETA + 2H₂O |
| R_PPA: | 7H₂ + 3CO → PPA + 3H₂O |
| R_BTA: | 9H₂ + 4CO → BTA + 4H₂O |
| R_PTA: | 11H₂ + 5CO → PTA + 5H₂O |
| R_HXA: | 13H₂ + 6CO → HXA + 6H₂O |
| C6toC3: | H₂ + HXA → 2PPA |
| C5toC2C3: | H₂ + PTA → ETA + PPA |
| C4toC1C3: | H₂ + BTA → CH₄ + PPA |

The ranges of input parameters for which the model is expected to be valid as listed in Table 3. Experimentation had been performed showing the performance for a range of temperature, pressure, H₂:CO, CO₂ concentration, and W/F. However, only a few data points were available to show the effect of pressure, and no data was available to show the effect of water. Given the importance of the water-gas shift reaction in methanol synthesis, this is a cause for concern. If water is present in the gas stream then there may be differences in the performance.

TABLE 3
Ranges of validity for the kinetic model

| Variable | Minimum | Maximum | Notes |
|---|---|---|---|
| Pressure | 3.1 MPa | 5.1 MPa | Almost all the |
| | | | experiments were at |
| | | | 5.1 MPa. Only 1 |
| | | | experiment at P = 3.1 |
| | | | MPa and another at |
| | | | P = 4.1 MPa were |
| | | | reconciled. |
| Temperature | 300° C. | 400° C. | |
| Syngas H₂:CO ratio | 0.7 | 3.7 | |
| Water | 0 | 0 | Inlet H₂O = 0 in all |
| | | | sets. The new |
| | | | information |
| | | | with nonzero water in |
| | | | the inlet was not |
| | | | reconciled due to |
| | | | problems with the |
| | | | data. |
| Inlet CO₂ molar fraction | 0 | 0.16 | |
| W/F, g h/mol | 1.87 | 17.8 | Most of the data at |
| | | | W/F = 1.87 g h/mol |

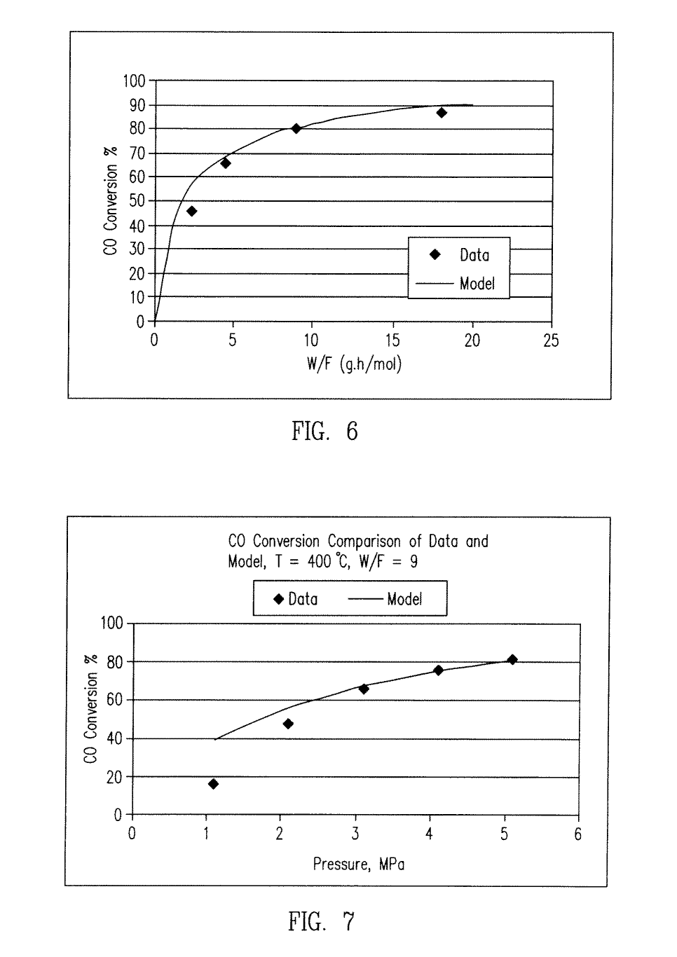

FIG. 6 shows the carbon monoxide conversion versus the reactor space velocity, in terms of W/F, which is the amount of catalyst divided by the molar feed gas flowrate. The data is for the pressure at 5 MPa and temperature of 400° C. The kinetic model fit of the carbon monoxide conversion versus WIF is a reasonably good fit.

FIG. 7 shows the carbon monoxide conversion versus the reaction pressure. Between 3 MPa and 5 MPa the fit is good. Below 3 MPa the predicted conversion is higher than what the experimental data shows. Of course less than 3 MPa was already suggested to be outside the region where the model is accurate.

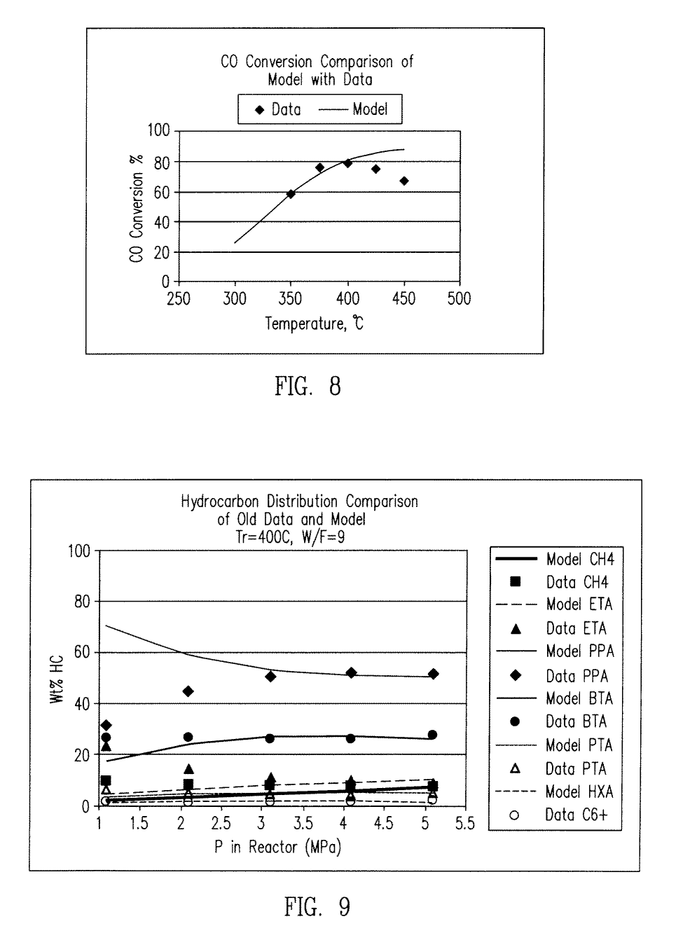

FIG. 8 shows the comparison of the kinetic model and the experimental data by carbon monoxide conversion versus the temperature curves. Between 350° C. and 400° C. the model is a reasonable fit. Above 400° C. the model conversion is higher than the experimental.

FIG. 9 shows the trend of individual hydrocarbons production versus the pressure at a temperature of 400° C. The model fits the data fairly well in numerical terms from 3-5 MPa. Below 3 MPa the model does not fit data. Numerical values for ethane, propane, and butane vary from model. Furthermore, the trend of C3 and C4 selectivity with pressure is the wrong direction.

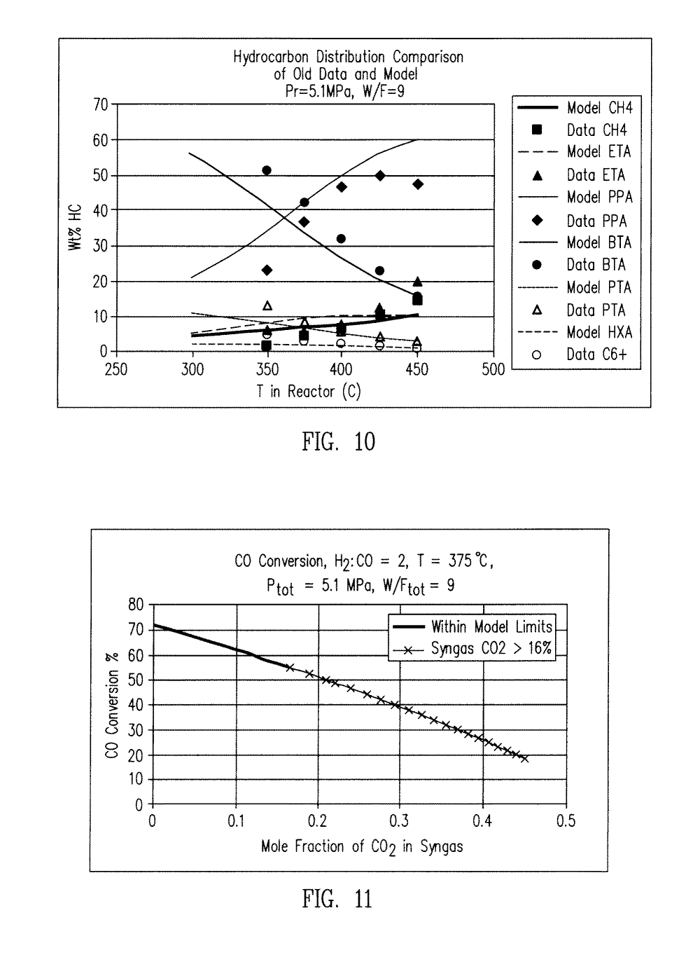

FIG. 10 shows the comparison of the model and data versus the reaction temperature at P=5.1 MPa and W/F=9 g h/mol. Trends for most of the components are reasonable. The data shows that for ethane the selectivity increases with temperature while the model shows an increase only up to 400° C. with leveling off afterwards. The numerical values of the component selectivities are best between 350-400° C.

A sensitivity analysis of the carbon monoxide conversion versus carbon dioxide concentration in the syngas feed showed the model predicts carbon monoxide conversion decreases somewhat with CO₂ in recycle stream. FIG. 11 shows the plot of the carbon monoxide conversion versus the carbon dioxide in the feed stream. Between zero and 16% CO₂ addition, the conversion decreases from over 70% to about 55%.

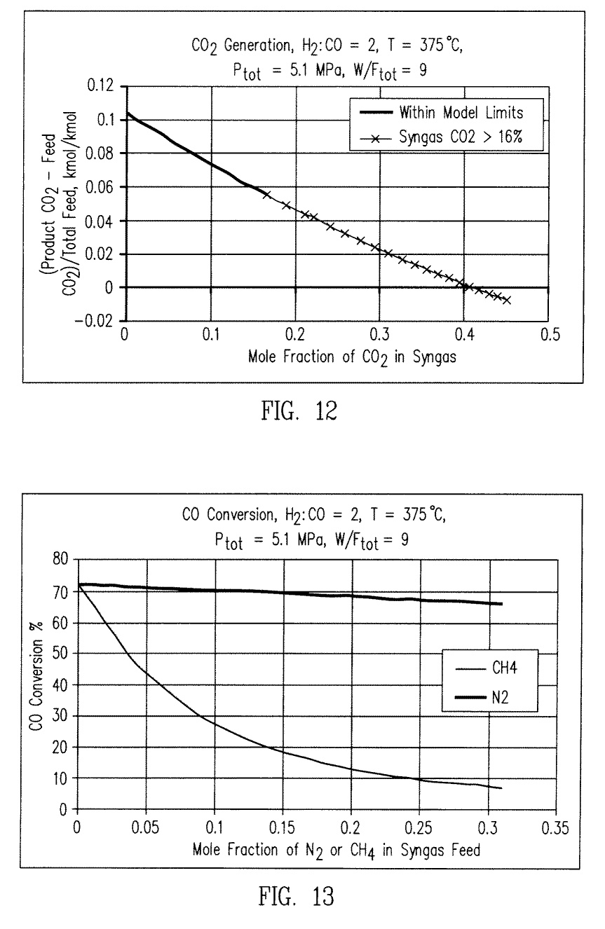

FIG. 12 plots the carbon dioxide conversion versus the carbon dioxide in the feed stream. If the model is extrapolated outside the range recommended for CO₂ concentration, then the predicted CO₂ concentration at the point of zero net carbon dioxide generation is about 40 mol %. It is believed that the molar concentration of CO₂ at equilibration may be in the range of 20-40%.

A sensitivity study was performed with respect to inert gases on the kinetic reaction model. Results of this study are illustrated in FIG. 13. For the effect of nitrogen in the feed, a small decrease in the carbon monoxide conversion is observed as the concentration of nitrogen in the feed increases. This is expected as the nitrogen concentration is increased the partial pressure of reactants is decreased. However the case of methane gave unexpected results. With methane addition, the kinetic model showed rapid decrease in the carbon monoxide conversion. With only 5 mol % methane, the conversion was predicted to decrease from over 70% to a little over 40%. By 25% methane feed addition the conversion was predicted to decrease to less than 10%. It is noted that the hydrocarbon formation reactions in the kinetic model contain a total alkane inhibition term in the kinetics equation. This total alkane inhibition included methane as an inhibiting component. The reason methane was included in the inhibition term of the kinetic model is that the experimental information available was not sufficient to be able to uniquely distinguish the effect of individual alkanes. However from the viewpoint of the chemistry, strong methane inhibition is not an expected phenomenon.

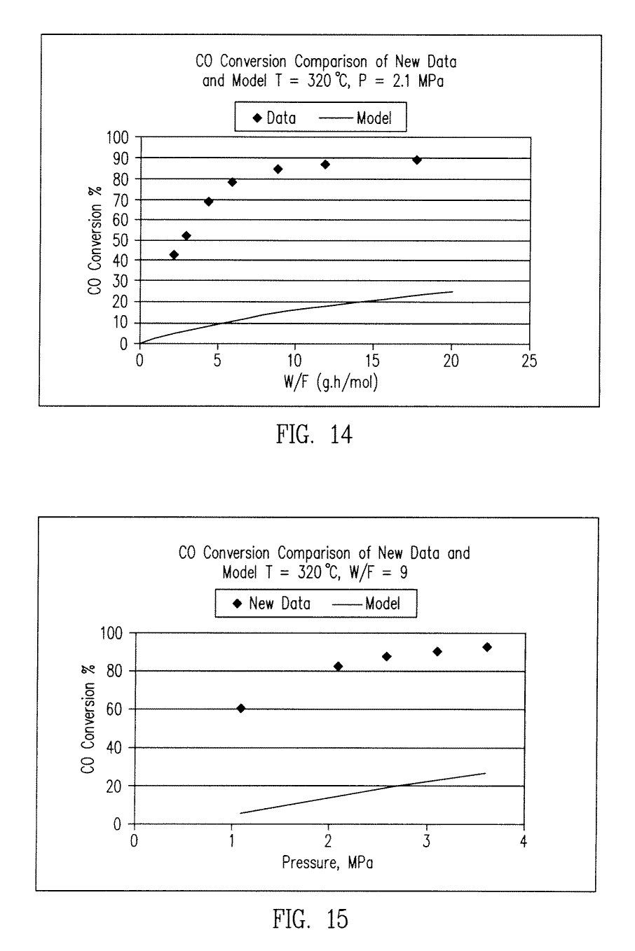

FIG. 14 shows the conversion of the catalyst as described in U.S. Pat. No. 7,297,825 versus W/F. With this catalyst the conversion of carbon monoxide is very high at much lower temperature and pressure than the original catalyst. FIG. 15 gives the carbon monoxide conversion of the new catalyst and the kinetic model versus reaction pressure. Note that the kinetic model, which is regressed from data of the old catalyst, had a tendency to overpredict the conversion at low pressure.

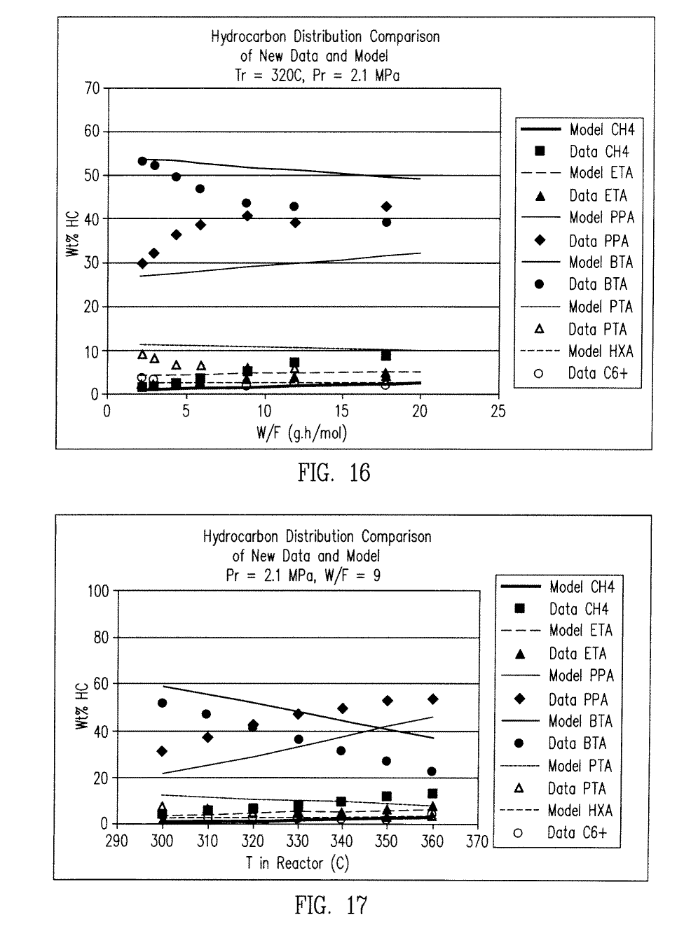

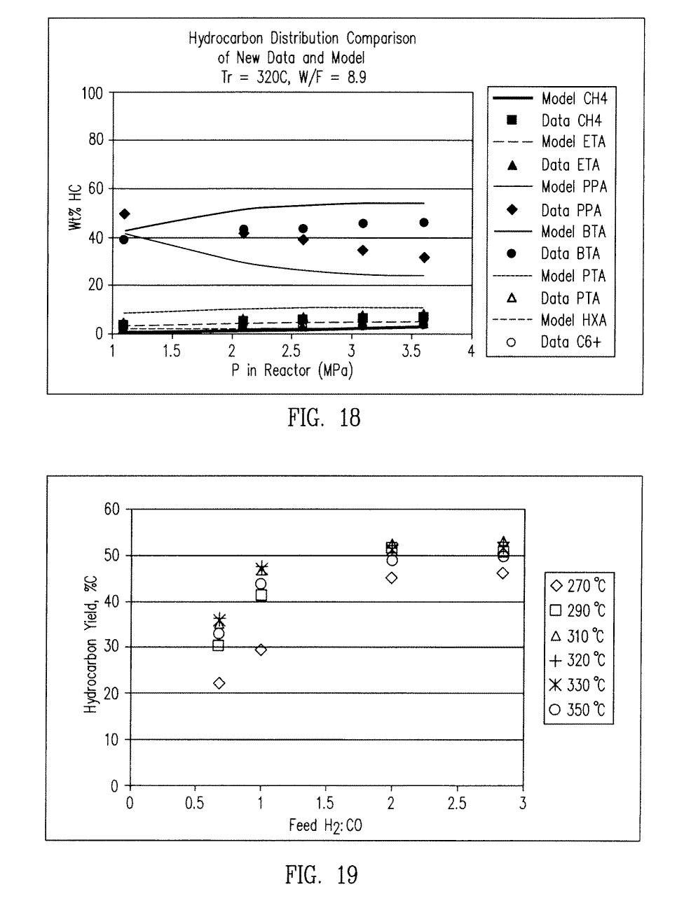

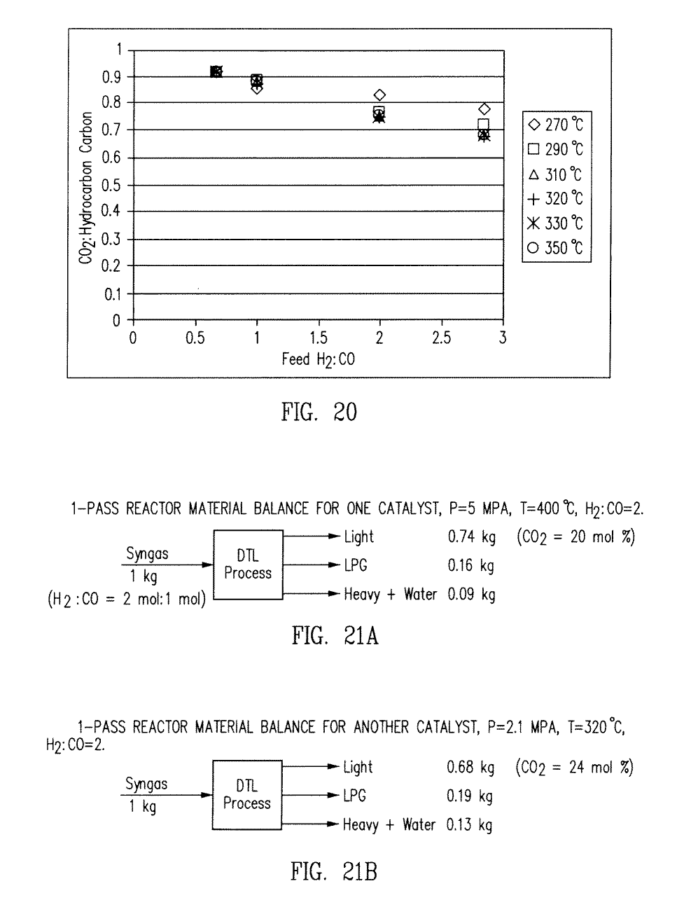

FIGS. 16, 17, and 18 show the hydrocarbon product distribution versus W/F, temperature, and pressure, respectively. Interestingly, the model correctly predicts the trends in propane and butane selectivity with pressure, where it did not for the data set that is was based on. Experimentation on varying the hydrogen to carbon monoxide ratio (H₂:CO) yielded an interesting result as shown in FIG. 19. As the ratio is increased, the hydrocarbon yield also increased. However this effect diminished after the ratio was above 2.0, as can be seen in FIG. 20, which shows the experimental data versus feed H₂:CO. On the other hand, the ratio of (carbon converted to carbon dioxide):(carbon converted to hydrocarbons) continued to decrease as the H₂:CO was increased.

FIG. 21A shows the overall material balance of a LPG reactor according to some embodiments of the present invention. Data is based on the mass of each component. For every kilogram of syngas feed to the reactor 0.16 kg of LPG is formed, 0.09 kg of water and heavy hydrocarbons are formed, and the remaining 0.74 kg are either light ends of unconverted syngas.

The light ends are 20 mol % carbon dioxide, indicating a large amount of carbon dioxide is formed in the reactor. The light ends may be recycled, and/or optionally one may convert the carbon dioxide back into carbon monoxide reactant or recycle it at a concentration that it is equilibrated in the reaction system.

FIG. 21B gives the material balance of the LPG reactor for the new catalyst under the conditions of P=2.1 MPa, T=320° C., H₂:CO=2, and W/F=8.9. Under these conditions the 1-pass conversion of carbon monoxide in the LPG reactor is over 90%. This catalyst produces 0.13 kg of water and heavy hydrocarbons, 0.19 kg of LPG, and 0.68 kg of light ends and unconverted reactants. The light ends in this case are 24 mol % carbon dioxide. The 1-pass yield of hydrocarbon products, both gas and liquid, on a carbon basis as the percent of carbon converted to hydrocarbon products, is over 50%. The 1-pass yield of liquid hydrocarbons C3 and heavier is nearly 46%.

Syngas is the feed to the LPG reactor 104. Syngas is a mixture of hydrogen and carbon oxides that is produced by reforming of a hydrocarbon feedstock material, in this case natural gas. Because it is not economical to transport syngas except by pipe and it is not readily available as a commodity chemical, syngas is normally produced via an on-site on-purpose syngas production unit, called a reformer. Furthermore, the LPG reactor generates methane and ethane byproducts that could be recycled as additional feedstock for the reformer or as fuel for the reformer if it is a fired heater type.

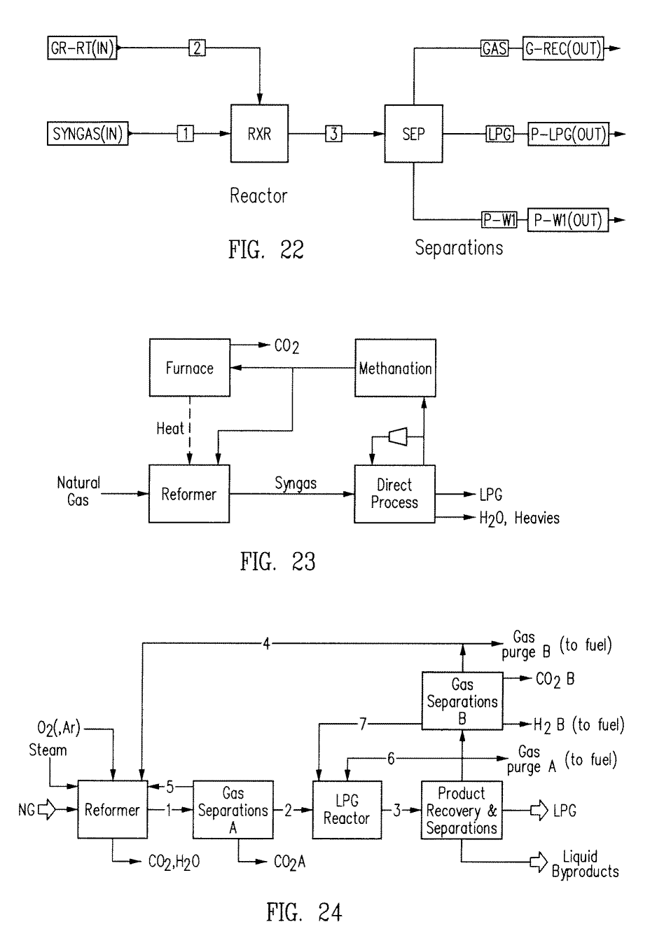

In the hierarchical design method, chemical plants are conceptually thought of as plant complexes consisting of a reactor and a separations system. Such a system is pictured in FIG. 22. With inclusion of the reformer in the analysis, this process already becomes a multi-plant complex with multiple reactors and separation systems. One can also anticipate that other types of reactions can be combined with the overall system of the present invention to improve process performance. One such possibility is the methanation reaction. In methanation, carbon oxides and hydrogen are converted to methane and water via the following overall reactions:

CO+3H₂→CH₄+H₂O

CO₂+4H₂→CH₄+2H₂O

Methanation can be used to reduce the amounts of carbon oxides that are recycled. If excess hydrogen is present, it can be used in combination with carbon dioxide to reduce carbon dioxide emissions. An example of combination of methanation with the system of the present invention is given in FIG. 23. In this embodiment, methanation is used to reduce the amount of hydrogen and carbon oxides that are recycled to the reformer.

The inventors have developed a basic superstructure for this process that covers a variety of system configuration embodiments. These are configurations for mass recycle streams only. One example of some embodiments of the present invention are illustrated in block flow diagram in FIG. 24. Note that not all configurations utilize all the blocks. For some embodiments certain blocks may do no processing and certain stream flows will actually be zero.

Table 4 shows descriptions of the alternative embodiments. Note that these are basic alternative variations and that options listed in the table can be combined to produce additional alternatives.

TABLE 4
Component flows present for different process alternatives

| | Stream 2 | | | | Stream 4 | | | | Stream 5 | | | | Stream 7 | | | |
|---|---|---|---|---|---|---|---|---|---|---|---|---|---|---|---|---|
| Option | H₂ | CO | CH₄ | CO₂ | H₂ | CO | CH₄ | CO₂ | H₂ | CO | CH₄ | CO₂ | H₂ | CO | CH₄ | CO₂ |
| Base* | ◯ | ◯ | ◯ | ◯ | ◯ | ◯ | ◯ | ◯ | | | | | | | | |
| A1 | ◯ | ◯ | ◯ | | ◯ | ◯ | ◯ | ◯ | | | | See | | | | |
| | | | | | | | | | | | | A1a-A1c | | | | |
| A1a | ″ | ″ | ″ | | ″ | ″ | ″ | ″ | | | | ◯ | | | | |
| A1b | ″ | ″ | ″ | | ″ | ″ | ″ | ″ | | | | | | | | |
| A1c | ″ | ″ | ″ | | ″ | ″ | ″ | ″ | | | | ◯ | | | | |
| A2 | ◯ | ◯ | | | ◯ | ◯ | ◯ | ◯ | | | ◯ | ◯^{⊥} | | | | |
| B1 | ◯ | ◯ | ◯ | ◯ | | ◯ | ◯ | ◯ | | | | | ◯ | | | |
| B1a | ″ | ″ | ″ | ″ | | ″ | ″ | ″ | | | | | ″ | | | |
| B1b | ″ | ″ | ″ | ″ | | ″ | ″ | ″ | | | | | ″ | | | |
| B2 | ◯ | ◯ | ◯ | ◯ | | | ◯ | ◯ | | | | | ◯ | ◯ | | |
| B3 | ◯ | ◯ | ◯ | ◯ | | ◯ | ◯ | | | | | | | | | |
| B4 | ◯ | ◯ | ◯ | ◯ | ◯ | ◯ | ◯ | | | | | | | | | |

| | | | | | | Gas Sep A | Gas Sep B |
|---|---|---|---|---|---|---|---|
| Option | Purge A | Purge B | CO₂ A | CO₂ B | H₂ B | Config. | Config. |
| Base* | ◯ | | | | | none | none |
| A1 | ◯ | | | | | CO₂ | none |
| | | | | | | Absorption | |
| A1a | ″ | | | | | CO₂ | ″ |
| | | | | | | Absorption | |
| A1b | ″ | | ◯ | | | CO₂ | ″ |
| | | | | | | Absorption | |
| A1c | ″ | | ◯ | | | CO₂ | ″ |
| | | | | | | Absorption | |
| A2 | ◯ | | | | | CO₂ | none |
| | | | | | | Absorption | |
| | | | | | | and/or PSA, | |
| | | | | | | Membrane, | |
| | | | | | | Cryogenic | |
| B1 | | ◯ | | | See | none | PSA, Cryogenic |
| | | | | | B1a-B1b | | or Membrane |
| B1a | | ″ | | | | ″ | PSA, Cryogenic |
| | | | | | | | or Membrane |
| B1b | | ″ | | | ◯ | ″ | PSA, Cryogenic |
| | | | | | | | or Membrane |
| B2 | | ◯ | | | | none | PSA, Membrane, |
| | | | | | | | or Cryogenic |
| B3 | | ◯ | | | | none | CO₂ Absorption |
| | | | | | | | and/or PSA, |
| | | | | | | | Membrane, |
| | | | | | | | Cryogenic |
| B4 | ◯ [symbol] | | | ◯ | | none | CO₂ Absorption |

*Water removal from syngas is already assumed to be beneficial in the base case
^{⊥}A1 CO₂ removal and recycle sub-options (A1a-A1c) can be repeated for A2 [symbol]
Reason for using Purge A is to reduce flow to CO₂ removal unit (No reason to remove CO₂ from tail gas)

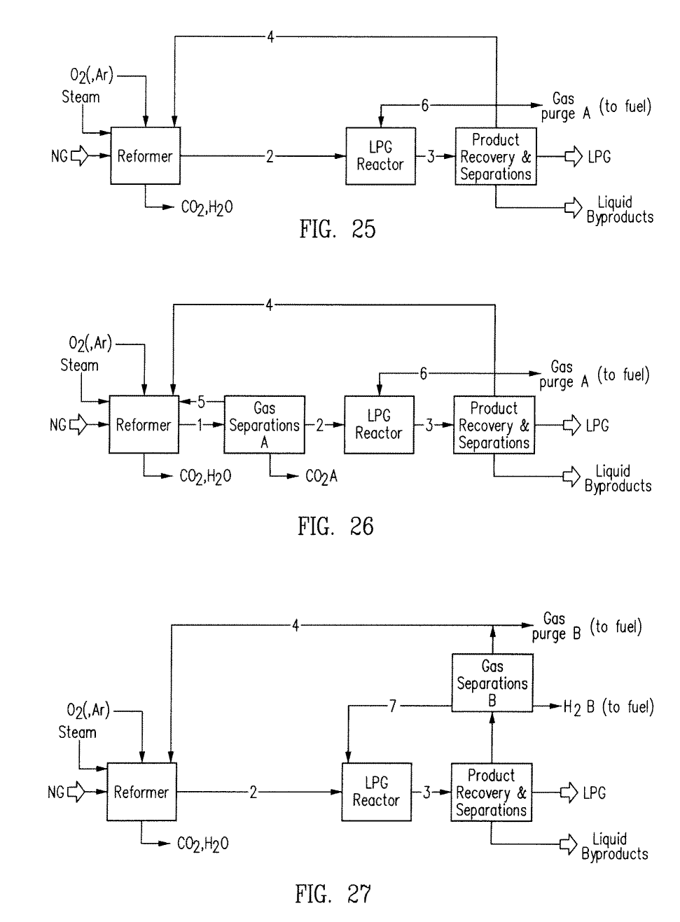

FIG. 25 shows a block flow diagram for other embodiments of the system and method according to one embodiment of the invention. In the base case there are no gas separations employed and only water and hydrocarbon recovery and fractionation are included in the separation system. This case is may be low in capital cost, however that determination is dependent on the flowrate of the recycle through the system. The disadvantages are that if light ends are recycled to the reformer then unconverted hydrogen also will be recycled. Hydrogen, which increases the gas volume of the reformer feed, may be consumed to form water if it is an oxygen-fed reformer (wasting both hydrogen and oxygen if the heat of reaction is not needed), and shifts the chemical equilibrium to reduce the conversion of methane in the reformer. The other disadvantage comes if light ends are recycled to the LPG reactor (Option C). In the LPG reactor loop, the methane and inert gases will accumulate, lowering the partial pressure of reactants and increasing the gas volume of the recycle loop and increasing the gas feed to the reactor, thus increasing its size and cost. Furthermore high concentrations of light gases increase the cost of LPG recovery in the separation system. The other consequence of recycling directly to the LPG reactor is that carbon dioxide will accumulate until it is equilibrated in the LPG reactor. This phenomena can be desirable in some instances for limiting the net CO₂ production.

FIG. 26 shows the block flow diagram for Embodiment A. With Embodiment A, gas separations are performed between the reformer and LPG reactor steps. There are two sub-options. Embodiment A1 comprises the step of remove and/or recycle CO₂ from syngas before feeding LPG reactor. CO₂ removal can be performed by absorption, typically using an amine solvent. Embodiment A2 comprises the step of removes and/or recycles CO₂ and CH₄ from syngas before feeding LPG reactor. This can be performed by absorption and/or pressure swing adsorption (PSA), membranes, or cryogenic separation.

FIG. 27 gives the flowsheet for Embodiment B. With Embodiment B there are 4 main sub-options. Embodiment B1 separates hydrogen from the reactor effluent gas for recycle to the LPG reactor. This can be performed by PSA, membrane, or cryogenic processing. Embodiment B2 separates H₂ and CO from the reactor effluent gas for recycle to LPG reactor by PSA or cryogenic processing. Embodiment B3 separates H₂ and CO₂ from the reactor effluent gas for recycle to LPG reactor by absorption and/or PSA, membrane, or cryogenic separation. Embodiment B4 separates CO₂ from the LPG reactor effluent gas and purges it.

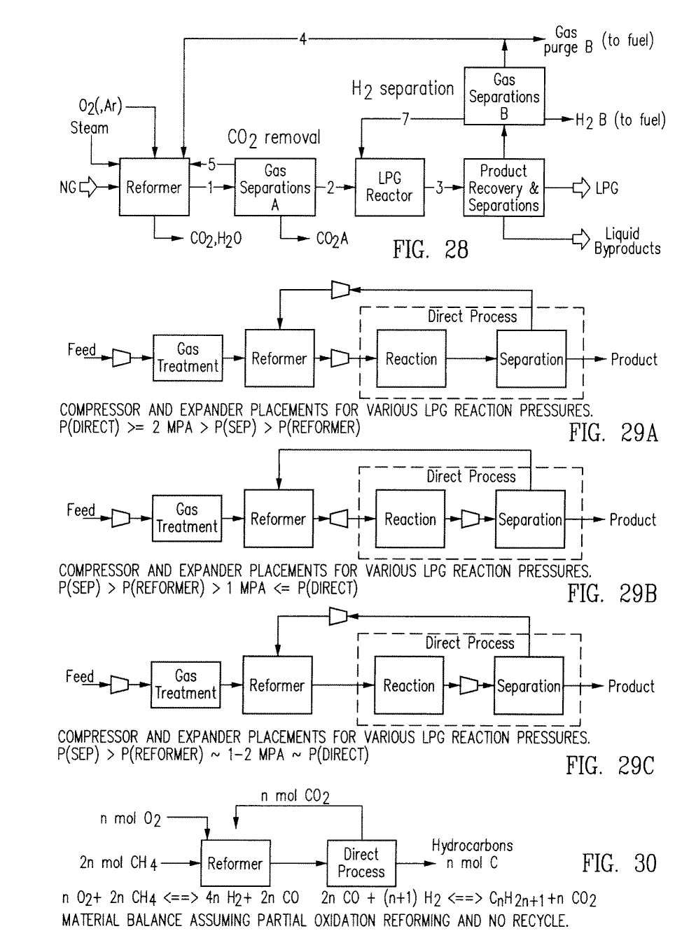

FIG. 28 illustrates a flowsheet for the process with a combination of the A1 and B1 embodiments. This is embodiment A1+B1. This configuration gives the benefits of carbon dioxide and hydrogen control for various points in the process. This allows the control of carbon dioxide by a combination of purging the separated carbon dioxide and by recycling to the reformer. It controls the H₂:CO ratio of the LPG reaction by the recycle of hydrogen. Since the hydrogen is separated from the tail gas, it does not need to be fed to the reformer, thus reducing the reformer load. This flowsheet can be used in the case that it is not cost effective to equilibrate carbon dioxide by recycling to the LPG reactor.

In addition to the layout of various flows of the process, compressor placement is another important aspect of the gas processing facility. FIGS. 29A-29C show some compressor placements, which depend on the LPG reactor pressure and its relationship to the reformer pressure and the separations system pressure. Although there is some scope for variation in the operating pressure of both these units, for the initial flowsheet design we assume the reformer will operate at about 2 MPa and the separation system will operate at about 3 MPa. Conditions for reforming are normally favorable at low pressure, however minimization of differences in the pressure as the gas flows through the process will ultimately give lower power consumption by the compressors.

D. Interaction of the LPG Reactor and the Syngas Generation Reactor

Syngas is a mixture of hydrogen, carbon dioxide and carbon monoxide. In the syngas synthesis plant, hydrocarbons are converted to carbon oxides and hydrogen. The reformer takes the natural gas and a source of oxygen, either water air, carbon dioxide, or elemental oxygen. The reaction is endothermic if water or carbon dioxide is used as the oxygen source. In this case additional energy from fuel is needed in order to maintain the reactor temperature. The reaction is exothermic if pure oxygen is used. In order to balance the hydrogen to carbon ratio, additional carbon may also be imported in the form of carbon oxides. Excess water should also be removed from the product.

Various types of reactors are available, such as the steam reformer [SR], where the hydrocarbons are reacted with steam as an oxygen and hydrogen source, the partial oxidation reformer [POX] where the hydrocarbons are reacted with oxygen, autothermal reforming [ATR] which is a combination of the above two, dry reforming where carbon dioxide is the source of oxygen, and mixed reforming, which is a combination of the above. In one preferred embodiment, systems of the present invention utilize mixed reforming as a combination that feeds carbon dioxide in addition to other types of oxygen sources.

Different reformers will require different designs and operating points. One important aspect is the H₂:CO ratio at various points in the process. The H₂:CO at optimal reformer performance may not be the optimal H₂:CO for the LPG reactor performance. For different H₂:CO ratios selected for the inlet to the LPG reactor, best reformer design and operating parameters are different. These two units are interdependent and optimization of this process should optimize the cost of the sum of these two units. Below is described various embodiments of the reformer 102 useful in the present invention.

1. Steam Reforming

The steam reformer utilizes water in the form of steam as a source of oxygen as well as a source of hydrogen. The main overall reactions that take place in the steam reformer are summarized below.

$${CH}_{4} + {H}_{2} O \to CO + 3 {H}_{2} ( Δ {H}_{298 K} = + 49.3 kcal \text{/} mol )$$

$${C}_{n} {H}_{m} + {n H}_{2} O \to n CO + ( n + \frac{m}{2} ) {H}_{2}$$

These reactions are endothermic. An external heat source is needed to maintain the reaction temperature. Another important chemical reaction in reforming operations is the water-gas shift reaction.

CO+H₂O [symbol] CO₂+H₂

This reaction is reversible. By adjusting the amount of water, the catalyst, and reaction conditions of different sections of the reformer, the hydrogen to carbon ratio can be controlled somewhat. In a typical application the objective is to maximize the hydrogen production. Thus a typical design will operate to shift the reaction to the right as much as possible. In the case of natural gas to LPG, hydrogen is plentiful, so having a shift converter is not particularly advantageous. The inventors have discovered that process performance will be better without a shift converter.

Steam reformers typically operate at low pressure, 0.15 to 3.5 MPa and temperatures from 750 to 900° C. A catalyst is used for the reaction. The catalyst typically contains nickel and has low tolerance to sulfur. The sulfur content should be reduced to 0.5 ppm or less in order to reduce poisoning. The temperature and pressure for the base design case will be 860° C. and 2 MPa.

The amount of steam used can vary over a considerable range although it also has a large impact on the process economics and operability. Typically the steam to carbon ratio for a steam reformer is around 3.0. It can be as low as 1.8, however if it is lower than this fouling will occur in the reactor and heat exchangers. The upper limit is bounded by economic considerations due to the energy cost of producing the steam, which cannot be reclaimed from the reaction effluent easily. In most cases the steam to carbon ratio is less than 6.0.

As can be seen the hydrogen to carbon monoxide (H₂:CO) ratio produced by steam reforming of methane is at least 3 (more if CO is shifted to CO₂). This is higher than the ratio that is required for LPG. If a steam reformer is used for the Direct process, we must make considerations in the design. First, since the H₂:CO generated by the reformer is higher than that of the product, the excess hydrogen must be dealt with in some way. One way is to limit any recycle.

Another method is to perform the reverse water-gas shift reaction on the LPG recycle gas to convert the excess hydrogen and the carbon dioxide formed in the LPG reactor to carbon monoxide and water. However the H₂:CO is higher than necessary even before carbon dioxide formation. Thus in that case there still will be excess hydrogen. The excess hydrogen must be either separated and purged or reacted with an additional source of carbon monoxide.

2. POX Reforming

In partial oxidation reforming, the methane reacts with a sub-stoichiometric amount of oxygen to produce a mixture of hydrogen and carbon oxides. Due to the absence of catalyst, a small amount of carbon formation is tolerated, and the reaction can be carried out at higher temperatures. The resulting syngas has lower H₂/CO ratios. In contrast to steam reforming, the POX reactions are exothermic.

$${CH}_{4} + \frac{1}{2} {O}_{2} \to CO + 2 {H}_{2} ( Δ {H}_{298 K} = - 8.5 kcal \text{/} mol )$$

$${C}_{n} {H}_{m} + \frac{n}{2} {O}_{2} \to n CO + \frac{m}{2} {H}_{2}$$

FIG. 30 shows an example assuming that partial oxidation is used for the reforming method. The overall reactions for the reformer and the LPG reactor are given below the corresponding block for the unit. The number of moles of CO₂ formed in the LPG reaction is what is experimentally shown for syngas H₂:CO of 2.0 or less. The reactions are multiplied by the factor needed to convert n moles of carbon into hydrocarbons. As can be seen, without recycle and reuse of material and energy, it will require 2n moles of CH₄ to convert n moles of carbon into hydrocarbon. One mole of carbon dioxide is formed for each mole of carbon converted to hydrocarbon. This simple material balance illustrates why partial oxidation reforming, although generating the H₂:CO close to the requirement of LPG, is not the optimal syngas generation method if CO₂ cannot be equilibrated within the LPG reactor.

Table 5 summarizes the conditions of the three types of reformers discussed above. These are the most common types of reforming operations.

TABLE 5
Comparison of typical reforming conditions

| | Steam | Partial | Autothermal |
|---|---|---|---|
| | Reforming | Oxidation | Reforming |
| External heat source | YES | NO | NO |
| needed | | | |
| Dedicated oxygen | NO | Desirable | Desirable |
| supply | | | |
| Capacity Limit | Small-Medium | Large | Large |
| Temperature range | 750-950° C. | 1200-1600° C. | 850-1000° C. |
| Pressure range | 1.5-35 atm. | up to 150 atm. | 2-4 atm. |
| Special requirements | Low Sulfur | — | — |
| Product H₂:CO | 3 | 2 | 2-3 |
| (for a CH₄ feed) | | | |

3. Dry Reforming

With dry reforming, carbon dioxide is used as the oxygen source. The overall reaction for the reaction of methane is:

CH₄+CO₂→2CO+2H₂ (ΔH_{298K}=+59.1 kcal/mol)

This reaction is more endothermic than with steam reforming. Also it produces a hydrogen to carbon monoxide ration (H₂:CO) of 1.0. Because the LPG reaction produces large amounts of carbon dioxide, the dry reforming reaction will be advantageous to this process. This reaction is beneficial to the Direct LPG process for two reasons. Firstly, it provides a means of recycle of the carbon dioxide and conversion of carbon dioxide to reactants for the LPG reaction. Secondly it produces syngas with a lower H₂:CO than by other methods. By using it at least partially we can control the H₂:CO in the process, this eliminating waste and buildup of hydrogen. The disadvantage is that the reaction is the most endothermic of the reactions presented here.

4. Mixed Reforming

Mixed reforming according to the present invention is a combination that feeds carbon dioxide in addition to other types of oxygen sources. Note that there are other possibilities for multiple oxygen sources, such as autothermal reforming with steam and oxygen as previously mentioned, and sequential steam and partial oxidation reforming (also known as 2-step reforming), however these are variations to improve the same abovementioned processes and we use the term mixed reforming as a way to distinguish the use of carbon dioxide mixtures from other variations of traditional reforming processes.

Carbon dioxide is a byproduct that is formed in the LPG reaction and mixed reforming offers a way to utilize the carbon dioxide in a recycle stream. Additionally it is a way to adjust the H₂:CO ratio of the syngas to match that of the LPG hydrocarbons that are being produced, slightly greater than 2.0. This process is more flexible than the dry reforming method.

Simulation and analysis was performed by using the experimental data as the prediction of the LPG reactor performance. Tables 6A and 6B show comparisons between data of different catalysts. For Embodiment A, the LPG reaction pressure was 5 MPa and temperature was 375° C. For Embodiment B the LPG reaction pressure was 2 MPa and temperature was 250° C. For both embodiments the W/F was 9 g h/mol. The embodiments had Embodiment B1 Flowsheet design, which included H₂ separation. 85% of remaining gas after H₂ removal is recycled to reformer. The reformer is fed oxygen and recycled CO₂. The separation systems were considered perfect separations. Recovery system gas feed pressure was 30 kg/cm². Because of the different reaction pressures there were some embodiment differences in compressor configuration. Embodiment A has a syngas compressor and reactor effluent turbine. Embodiment B has a reactor effluent compressor only.

The data shows that for both cases the reformer and the separations systems are about the same size for the same LPG production rate. The LPG reactor is somewhat larger for Embodiment B because of the gas volume due to the lower pressure. The power equipment requirement for embodiment B is only 60% of that for Embodiment A. It is further shown that Embodiment B has a higher carbon efficiency and lower utility use under the same flowsheet configuration.

TABLE 6A
Comparison of process with different catalyst performance.

| Process | | | Recovery & | Gas | Power |
|---|---|---|---|---|---|
| Area: | Reformer | LPG Reactor | Separations | Separations | Equipment |
| Case A | (Base Case) | (Base Case) | (Base Case) | (Base Case) | (Base Case) |
| Case B | about the same | somewhat larger | about the same | about the same | 60% of Case A |

TABLE 6B
Comparison of Embodiment A and Embodiment B.

| | | Variable | |
|---|---|---|---|
| | | Cost | Total Power |
| | Carbon | Function | Equipment |
| Case | Efficiency % | Yen/kg LPG | MW |
| Case A | 57 | 20.3 | 63.8 |
| Case B | 60 | 18.4 | 38.1 |

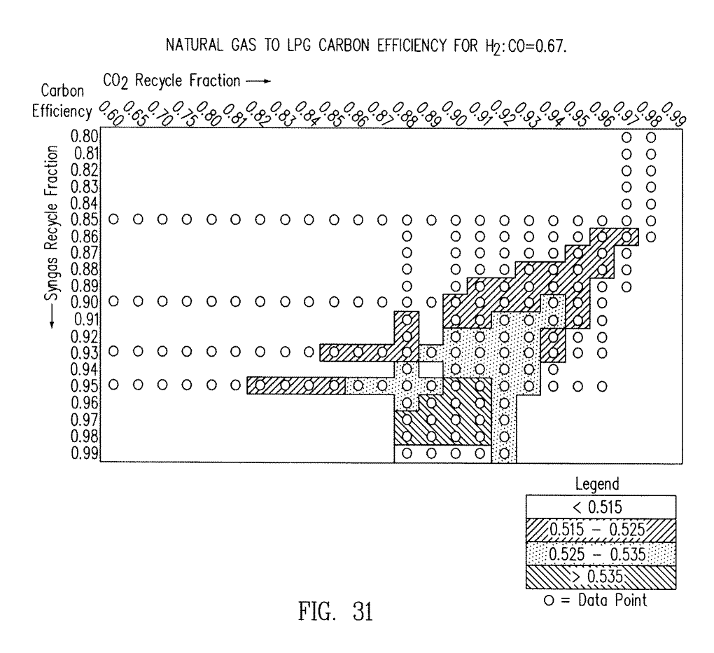

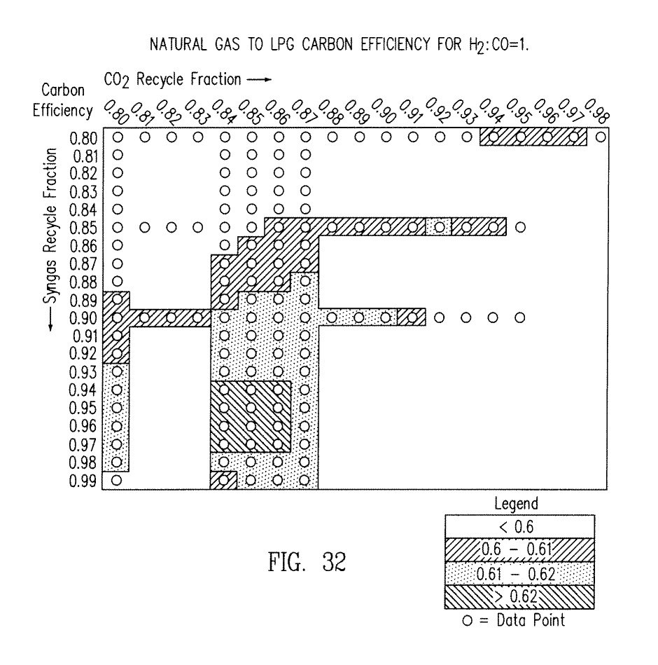

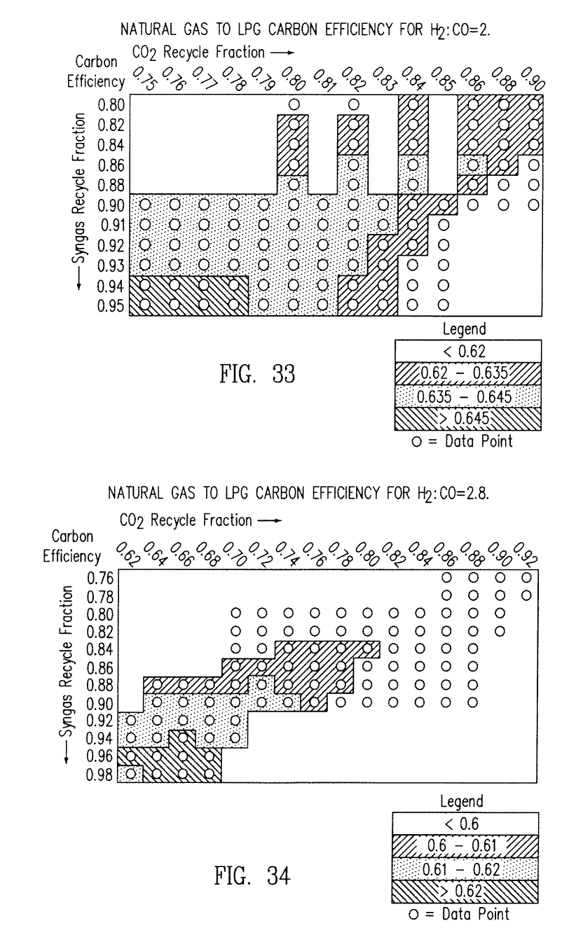

A sensitivity analysis was performed by varying the carbon dioxide recycle fraction and the syngas recycle fraction for the syngas H₂:CO values where data was available. FIGS. 31-34 give the natural gas to LPG carbon efficiency for H₂:CO of 0.67, 1.0, 2.0, and 2.8 respectively. The best carbon efficiencies were for H₂:CO of 2.0. This is about 65%. The total carbon efficiency, including all liquid hydrocarbons is about 5% higher than the LPG only carbon efficiency.

E. Separation System

This section describes the development of the separation system and shows some embodiments of the separation system according to the present invention. Table 7 lists the boiling points of major components. They range from hydrogen as the lightest, which is noncondensible for practical industrial purposes, to water. For separations of components heavier than methane, the boiling point difference is sufficient for fractionation by normal distillation for mixtures with no azeotropes present. Because of the low boiling points of methane and lighter components, cryogenic separations or alternate technologies such as membrane separations must be used to separate these components. For methane through butane, it is desirable to operate distillation columns at elevated pressure to minimize the cost of refrigeration for column condensers.

TABLE 7
Normal boiling points of major components in the LPG process.

| | Boiling Point |
|---|---|
| Component | ° C. |
| Hydrogen | −252.8 |
| Nitrogen | −195.8 |
| Carbon Monoxide | −191.5 |
| Oxygen | −183.0 |
| Methane | −161.5 |
| Ethane | −88.6 |
| Carbon Dioxide | −78.5 |
| Propane | −42.0 |
| DME | −24.8 |
| i-Butane | −11.7 |
| n-Butane | −0.5 |
| n-Pentane | 36.1 |
| Methanol | 64.7 |
| n-Hexane | 68.7 |
| Water | 100.0 |

The separations pressure is also guided by an upper limit if vapor-liquid equilibrium is to be used as a recovery or separation principle. This applies to the recovery method and to separation by fractionation (distillation). If the pressure becomes higher than the critical pressure of the component, then it cannot be condensed. For example if the pressure is higher than 40 bar it may be difficult to design a column with butane as the bottoms product. Therefore the upper limit of the separations system pressure is around 40 bar. In typical applications, for example in ethylene plants etc. the optimal separation pressure at the beginning of the separation sequence is around 3 MPa. Table 8 shows critical properties of selected components:

TABLE 8
Critical properties of selected components.
Critical Properties

| Compound | T_{c}, ° C. | P_{c}, bar |
|---|---|---|
| Methane | −82 | 46 |
| Ethane | 32 | 49 |
| Carbon Dioxide | 31 | 74 |
| Propane | 97 | 43 |
| iso-Butane | 135 | 37 |
| n-Butane | 152 | 38 |

Table 9 shows the azeotropic behavior of the binary pairs of components in this process (Gmehling 2004). Most binary mixtures considered here do not have azeotropic behavior. However there are a few mixtures that we must make note of. One is that of carbon dioxide and ethane. Another is DME and propane. Another is DME and isobutane. Another trouble component is methane, which forms azeotropes with propane, isobutene, n-butane, pentane, hexane, and benzene. If aromatics are present they will form azeotropes with some components. Also water forms azeotropes with several of the LPG components.

TABLE 9
Azeotropic behavior of LPG process components

| | Nitrogen | Carbon Monoxide | Oxygen | Methane | Ethane | Carbon Dioxide | Propane |
|---|---|---|---|---|---|---|---|
| Nitrogen | | | | | | | |
| Carbon Monoxide | N | | | | | | |
| Oxygen | N | | | | | | |
| Methane | N | N | | | | | |
| Ethane | N | N | | N | | | |
| Carbon dioxide | N | N | N | N | Y | | |
| Propane | N | N | | N | N | N | |
| DME | | | | | | N | Y |
| i-Butane | N | | | N | N | N | N |
| n-Butane | N | | | N | N | N | N |
| n-Pentane | N | | | N | N | N | N |
| Methanol | | N | | N | N | N | Y; HET < |
| | | | | | | | 21° C. |
| n-Hexane | N | | | N | N | N | N |
| Benzene | | | | N | N | N | N |
| Water | N | | N | N | N | N | Y; HET |
| | | | | | | | high P |
| NO DATA | | | | | | | |

| | DME | i-Butane | n-Butane | n-Pentane | Methanol | n-Hexane | Benzene |
|---|---|---|---|---|---|---|---|
| Nitrogen | | | | | | | |
| Carbon Monoxide | | | | | | | |
| Oxygen | | | | | | | |
| Methane | | | | | | | |
| Ethane | | | | | | | |
| Carbon dioxide | | | | | | | |
| Propane | | | | | | | |
| DME | | | | | | | |
| i-Butane | Y | | | | | | |
| n-Butane | N | N | | | | | |
| n-Pentane | | | N | | | | |
| Methanol | N | Y | Y | Y | | | |
| n-Hexane | | | N | N | Y | | |
| Benzene | | | N | N | Y | Y | |
| Water | | | HET | HET | N | HET | HET |
| NO DATA | | | | | | | |

When azeotropes are present, simple distillation is inadequate for performing complete separations. In such cases azeotropic distillation methods must be considered, which require more than one distillation column to separate a binary mixture, or alternative separation method must be considered such as adsorption or membrane separations.

Water can be removed to a great extent by cooling, condensing, and decanting the liquid phases formed. For removal of the remaining water, molecular sieves can be used to adsorb water to low concentrations.

Regarding DME and methanol separations, it is recommended the LPG reactor be designed to minimize DME and methanol in the reactor outlet to avoid the need for separation of these components from hydrocarbons that exhibit azeotropic behavior. If molecular sieves are used to remove water, then some of these oxygenates may also be removed by the molecular sieve.

It is noted that if aromatics are formed, then they can become an important separations issue. As noted in Table 9 the azeotropes of benzene and other components of the system. Toluene has similar azeotropic forming conditions as benzene. Another concern with aromatics is the possibility of heavy aromatics formation, such as multi-methylated aromatics. These heavy compounds are capable of solid formation. For example pentamethyl benzene has a freezing point of 53° C. and hexamethyl benzene has a freezing point of 165° C. Solids formation may cause damage to compressor equipment and fouling of heat exchanger surfaces if not avoided. On the other hand, there is no data available showing the presence of aromatics as reaction products. Therefore aromatics cannot be considered at this point. If later aromatics are found to be present, the separation system should be redesigned to account for that circumstance. In general, if additional compounds are found to be present than what are considered here, we recommend the separation system be reconsidered in entirety to take the additional components into account.

Because of the large amounts of hydrogen and carbon dioxide in the reactor effluent, the concentration of LPG components in this stream is only a few percent. This is similar to the concentration of LPG components found in natural gas from the field. Therefore the emphasis of the separation system is on the recovery of LPG that can be achieved. The most common recovery methods for LPG recovery are cryogenic and by absorption.

In cryogenic recovery, the gas mixture is cooled to a low temperature to condense the liquefiable hydrocarbons. Then the condensed mixture is fractionated by distillation. In absorptive recovery, a solvent is used to absorb the LPG from the gas phase to the liquid phase. Additional processing separates the LPG and the solvent.

1. Description of One Preferred Embodiment

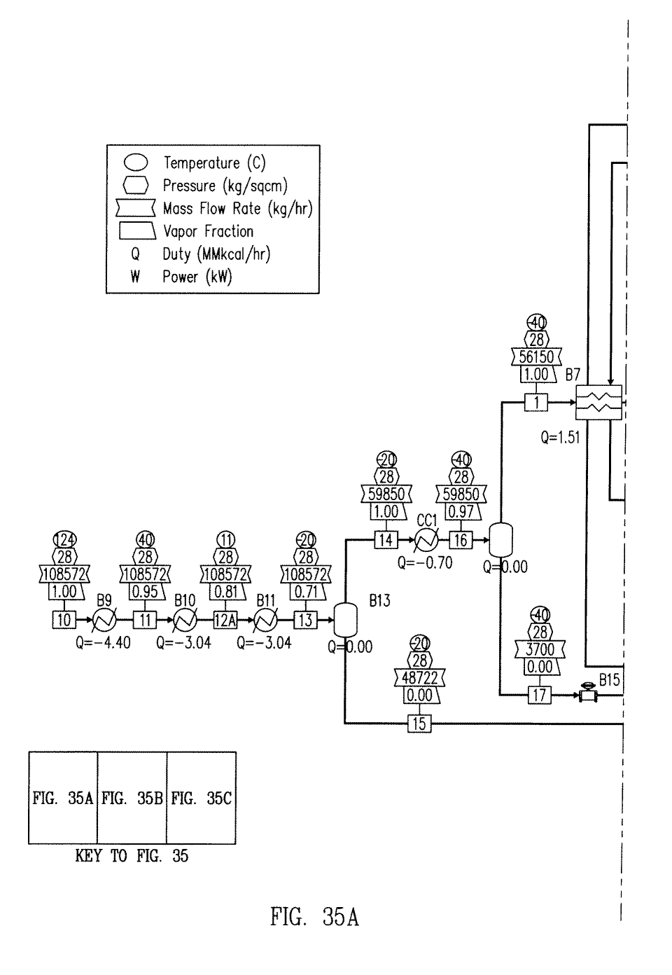

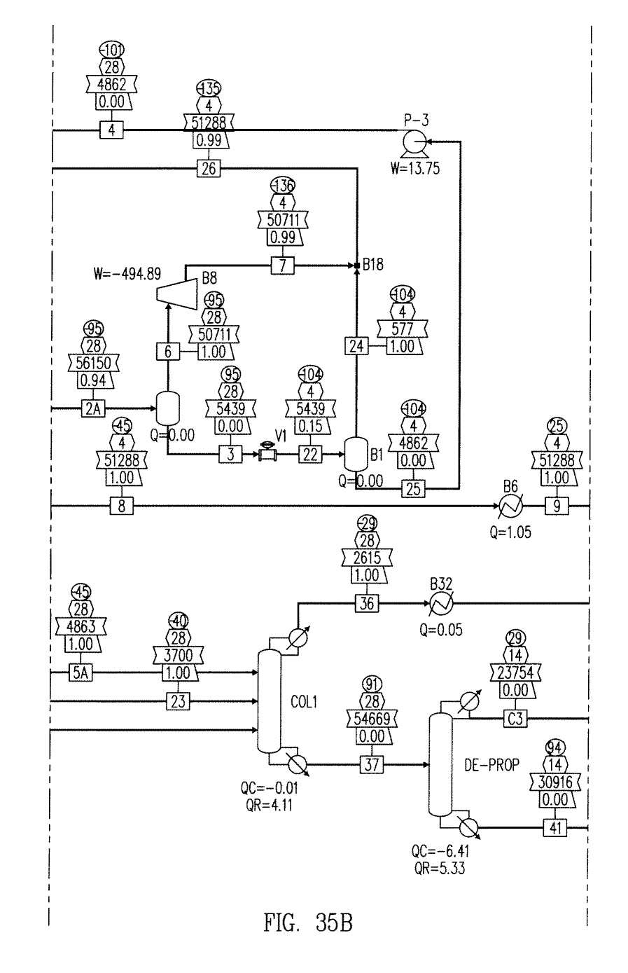

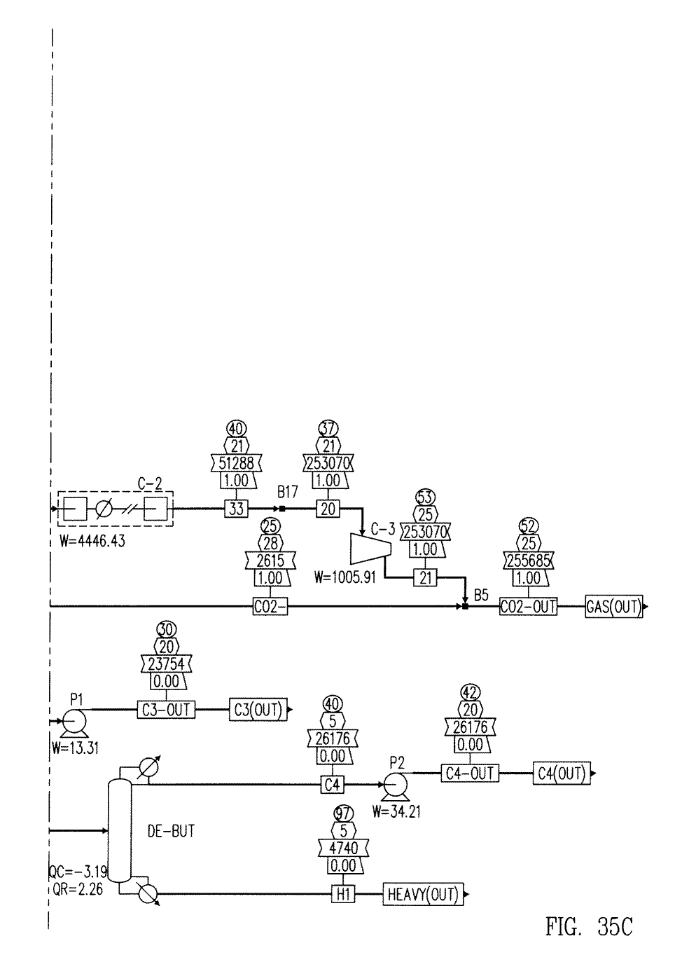

In some embodiments the separation system 106 is based on cryogenic recovery of LPG. In one example, the gas stream is first pretreated. Gas enters this section at a pressure near 3 MPa. After pretreatment, the gas is cooled in stages to condense the liquid hydrocarbons. The stages correspond to levels of the refrigeration cascade in the utility section of the plant. After each stage vapor-liquid separators collect the liquids. Further recovery is achieved by reducing the gas pressure to effect Joule-Thomson expansion that further cools the gas without external refrigeration. The liquid is sent to a distillation column that strips the light ends from the product hydrocarbons. The light ends recovered in the first column are mixed with the recycle stream. The condenser of this column requires some refrigeration to maintain its temperature. The overhead gas is recompressed for recycle to the plant. The bottoms of the first column contain the liquid hydrocarbons and are sent to additional fractionations. The first fractionation is the depropanizer which recovers the propane in the distillate. The second fractionation recovers butanes in the distillate and heavier compounds in the bottoms. Both of these columns operate at pressures such that the condenser temperature is in the range serviceable by cooling water. One example is shown in FIG. 35. This diagram shows the unit operations of the process. Actual equipment may appear differently and may combine more than one operation per equipment or divide an operation among several equipment units.

Table 10 gives the column specifications for the simulation according to some embodiments of the present invention. Table 11 gives some of the results and details for the distillation columns according to some embodiments of the present invention.

TABLE 10
Column specifications.

| | | | Number of | Top | |
|---|---|---|---|---|---|
| | | | Equilibrium | Pressure, | Feed |
| Column: | Specifications | | Stages | kg/cm² | Stage |
| Column 1 | Spec 1: | 99% recovery | 25 | 28 | 16 |
| | Spec 2: | of C3 in | | | |
| | | bottoms | | | |
| | | fraction of | | | |
| | | C3/(C3 + CO2) | | | |
| | | in bottoms | | | |
| | | stream = 0.999 | | | |
| depropanizer | Spec 1: | 1% of C4 in C3 | 30 | 14.3 | 15 |
| | Spec 2: | 0.8 mol % of | | | |
| | | C3/(C3 + C4) | | | |
| | | in bottoms | | | |
| debutanizer | Spec 1: | 0.8% mole | 24 | 4.7 | 12 |
| | Spec 2: | fraction of C5 | | | |
| | | in C4 99.9% | | | |
| | | recovery of | | | |
| | | C4 in distillate | | | |

TABLE 11
Distillation column details.

| | | | |
|---|---|---|---|
| Column: | Column 1 | Depropanizer | Debutanizer |

| | | | |
|---|---|---|---|
| Distillate Rate, kg/h | 2,805 | 23,552 | 26,276 |
| Bottoms Rate, kg/h | 54,575 | 31,024 | 4,747 |
| Top Temperature, ° C. | −39 | 32 | 40 |
| Bottom Temperature, ° C. | 92 | 94 | 97 |
| Top Pressure, kg/cm² | 28 | 14.3 | 4.7 |
| Reboiler Duty, MMkcal/h | 4.2 | 5.3 | 2.3 |
| Condenser Duty, MMkcal/h | 0.1 | 6.4 | 3.2 |
| Molar Reflux Ratio | 0.2 | 2.5 | 0.5 |
| Column Diameter, m | 2.6 | 2.4 | 1.6 |

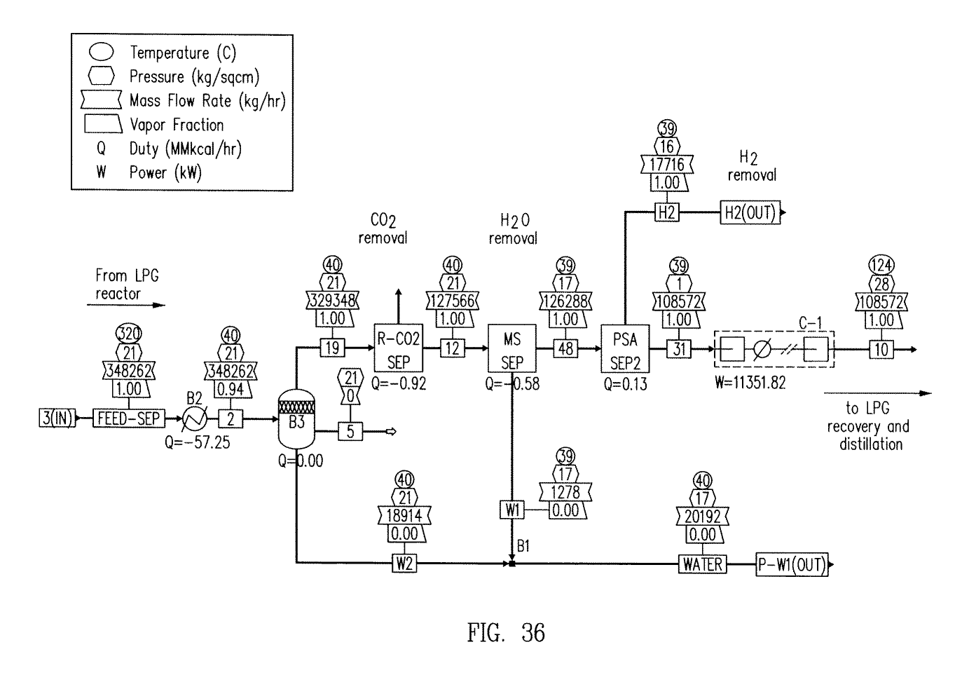

In some embodiments, gas pretreatment section is preferably used. The pretreatment section removes components that are not well handled by the LPG recovery system. It removes water. Water would form ice in the cryogenic recovery section. This also avoids azeotropic distillation to separate the water-butane azeotrope. The pretreatment section removes carbon dioxide. Carbon dioxide would solidify in the cryogenic LPG recovery section if not removed. Another advantage of CO₂ removal is reduction of the gas volume to the LPG recovery section. This helps to increase the LPG concentration and lower the capital and utility cost of subsequent sections. Finally, since the flowsheet selection designated hydrogen separation and recycle, hydrogen removal was placed in the pretreatment section. Removal of hydrogen before LPG recovery further reduces the gas volume to the LPG recovery section and raises the LPG partial pressure so the capital cost of the LPG recovery section is reduced. Water removal is after the carbon dioxide removal because the CO₂ absorption solvent is amine in aqueous solution. A block flow diagram of the pretreatment section as pictured by graphics from the process simulator according to some embodiments of the present invention is shown in FIG. 36.

2. Other Separation System Embodiments

Other embodiments are briefly evaluated in this section. The depropanizer and debutanizer columns were nearly the same whether the recovery was by cryogenic or absorptive recovery. This is because the production rate and feed composition was the same. The utility consumption of the absorber system was similar to the cryogenic system in terms of kilocalories of heating and cooling needed. Thus the absorber recovery system will differ mainly in capital cost due to the fact more columns are needed for that type. Thus the cryogenic LPG recovery is the preferred embodiment.

There are however circumstances that are possible that would incline the decision towards an absorptive LPG recovery. One would be actual formation of heavier byproducts. A cryogenic system would be more subject to fouling by heavy compounds. Another would be the elimination of carbon dioxide and hydrogen separation from the flowsheet. If the LPG reaction performance can be verified under CO₂ equilibration conditions then the process may be designed to operate without a CO₂ removal step. In that case LPG absorption without prior removal of CO₂ may be a good choice of design and alternate embodiment.

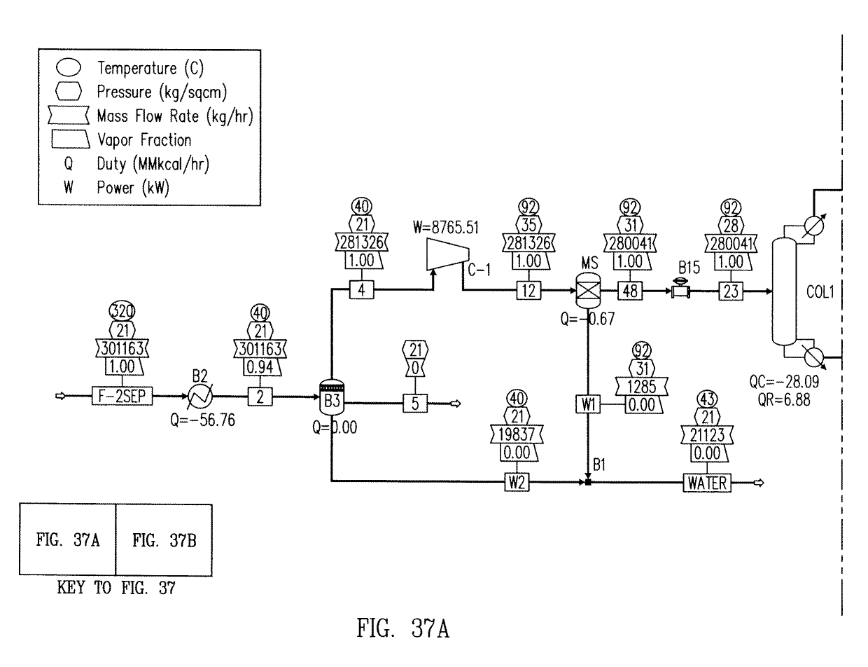

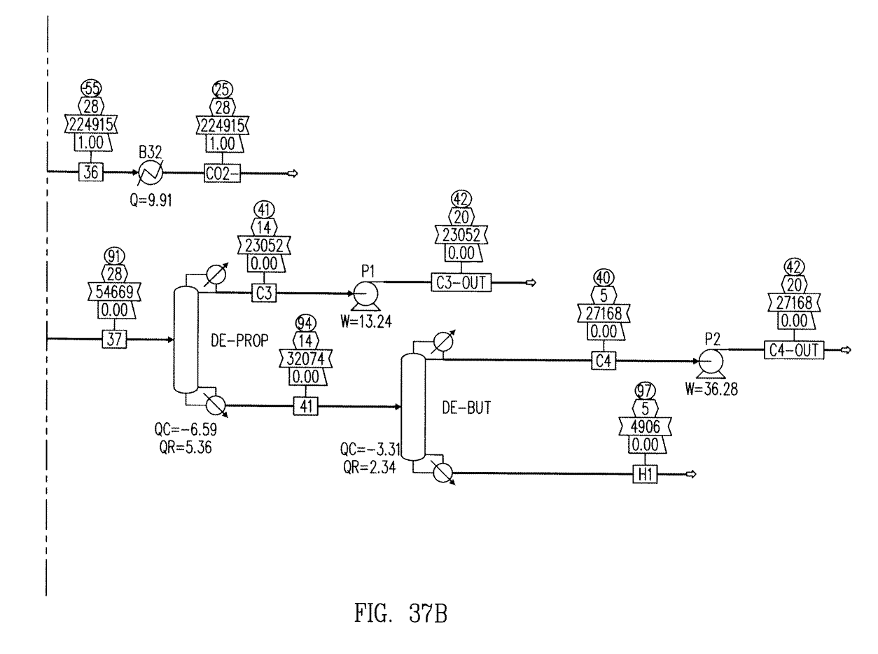

Another example of a separation system by cryogenic separation is given in FIG. 37. In this figure, the reactor effluent is cooled by heat recovery and cooling water. Some of the water vapor in the stream is condensed and is separated by vapor-liquid separation equipment. The gas is compressed to 35 kg/cm² and fed to a molecular sieve to further dehydrate the gas. Then the stream is fed to a distillation section. In the first column, the LPG is recovered in the bottoms stream. Carbon dioxide and lighter components exit as a vapor distillate. The condenser temperature is −55° C., a few degrees above the freezing point of carbon dioxide to prevent carbon dioxide solidification. The overhead gas can be recycled to the process. The bottoms product is fed to two additional columns, a depropanizer and a debutanizer, which recover the product propane and butanes as distillate products, respectively. The bottoms of the third column contains the heavy components. The pressures of the latter columns are adjusted so that the condensers operate at temperatures serviceable by cooling water.

The separation system described above is desirable because it is simple and only uses a few pieces of equipment. Apparently, there is some sophistication built in the design because it removes water first, thus avoiding water-hydrorcarbon azeotropes, the temperatures are maintained to prevent CO₂ solidification, the pressures are maintained to reduce utility consumption by refrigeration etc. Also, it was investigated the effect of precooling of the gas feed and was found that precooling did not reduce the utility consumption if the entire stream was fed to the column.

There are some difficulties with the system shown in FIG. 37. One is that the entire gas plus liquid feed enters the first column. The amount of gas is much larger than the amount of liquid. Thus the column would be difficult to operate and control and is likely to have large amounts of LPG lost due to liquid entrainment in the gas phase. In addition, the condenser duty calculated by the simulation was 28 MMkcal/h, which is a large condenser duty considering the LPG production rate and especially the refrigeration conditions of the condenser. The cost of this design would be very high in terms of utility and refrigeration equipment needed for the design.

Embodiments involving absorptive recovery, or at least partial absorptive recovery were simulated. Case studies with full LPG recovery by absorption were not simulated because it was estimated that large flows of absorption solvent were needed to obtain high recovery of the LPG. A few of the embodiments are discussed here.

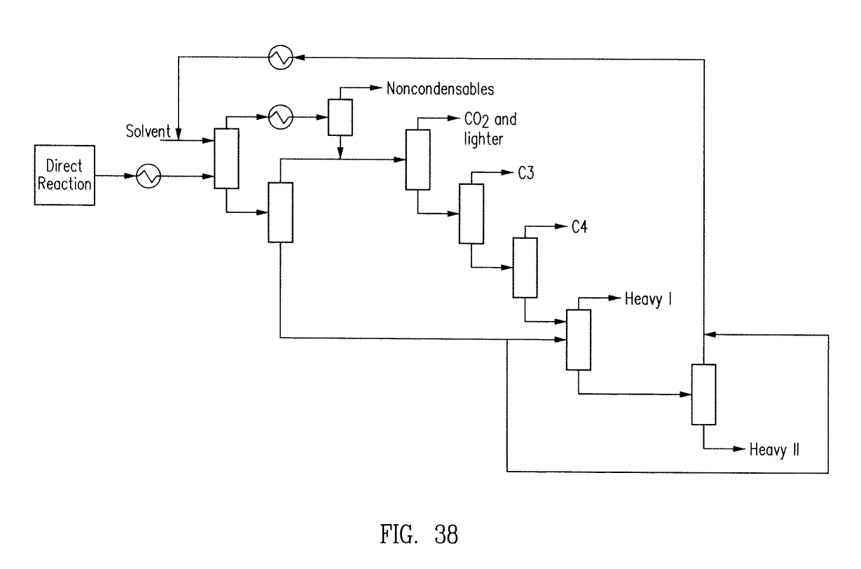

FIG. 38 illustrates a block flow diagram of a separation system that recovers heavy components by solvent recovery. The LPG components are then recovered by cryogenic recovery. The advantages of this embodiment are that it requires lower solvent circulation rates than by recovering LPG by absorption. Also by using an absorber heavy compounds can be removed first, avoiding the possibility of equipment fouling by the heavy compounds. In this design the reactor effluent is partially cooled for heat recovery purposes and subsequently fed to a quench/absorber tower to further cool the effluent and remove heavy components. The heavy components exit at the bottom with the absorption solvent. LPG and lighter components exit the top of the column. The overhead stream is then partially condensed to recover the LPG by cooling. The bottoms are fed to a stripper to strip LPG and lighter components. The LPG goes to a fractionation section. The remaining solvent is recycled, part of which is also processed in a solvent treating section to purge heavy components from the system. The recycled solvent must be cooled for further use. Typical solvents that can be used for this application are linear hydrocarbons in the C9-C12 range, or naptha.

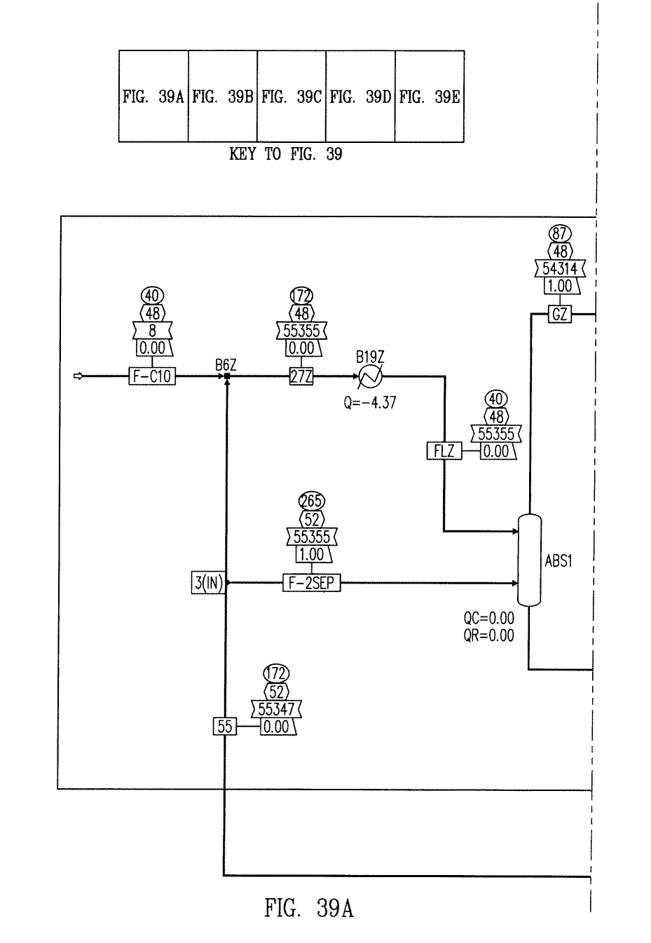

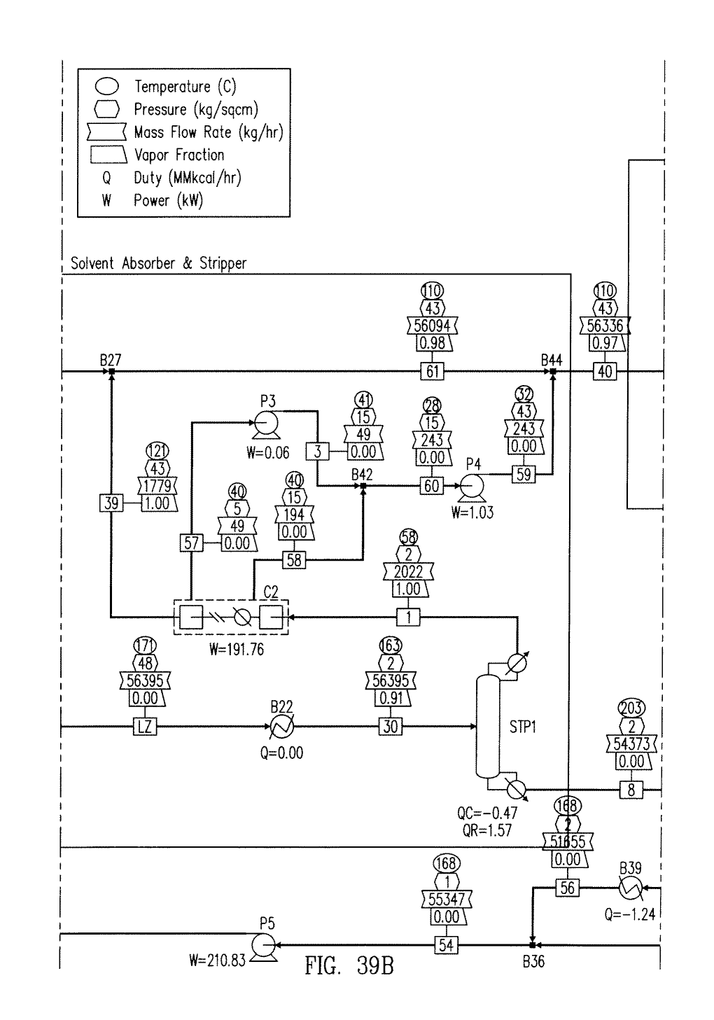

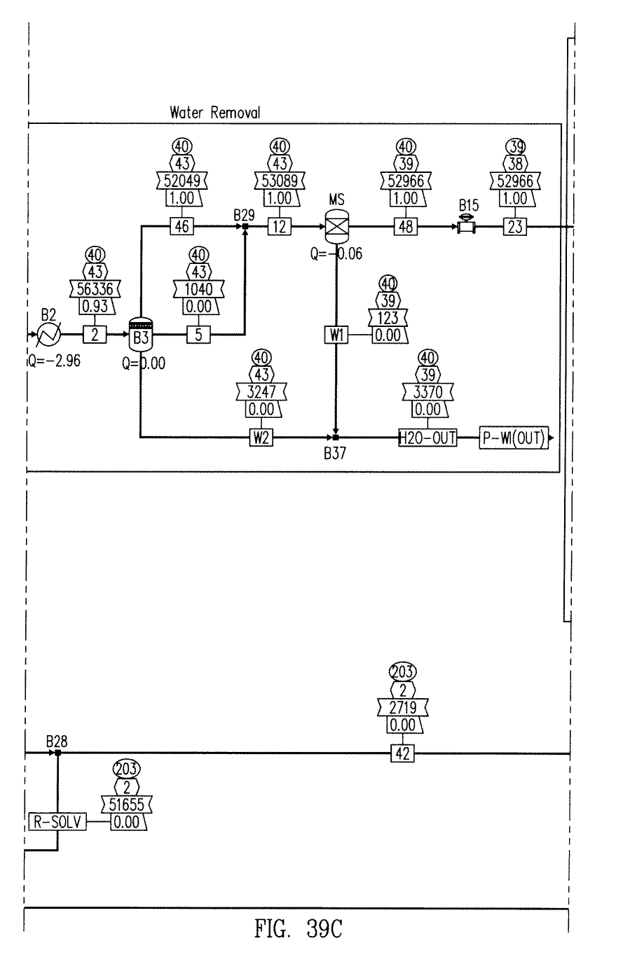

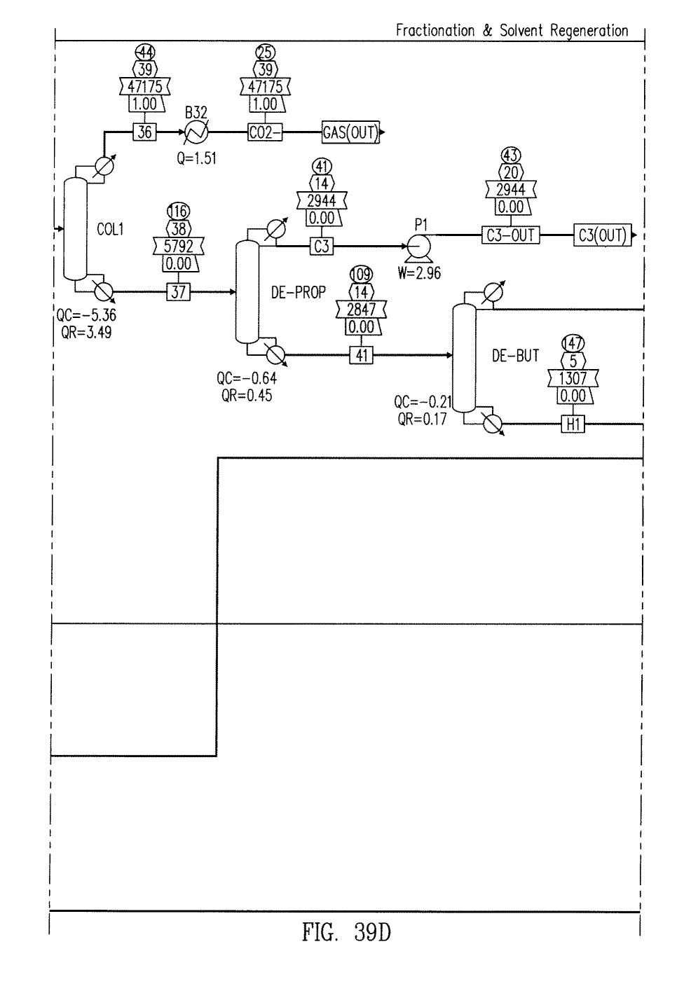

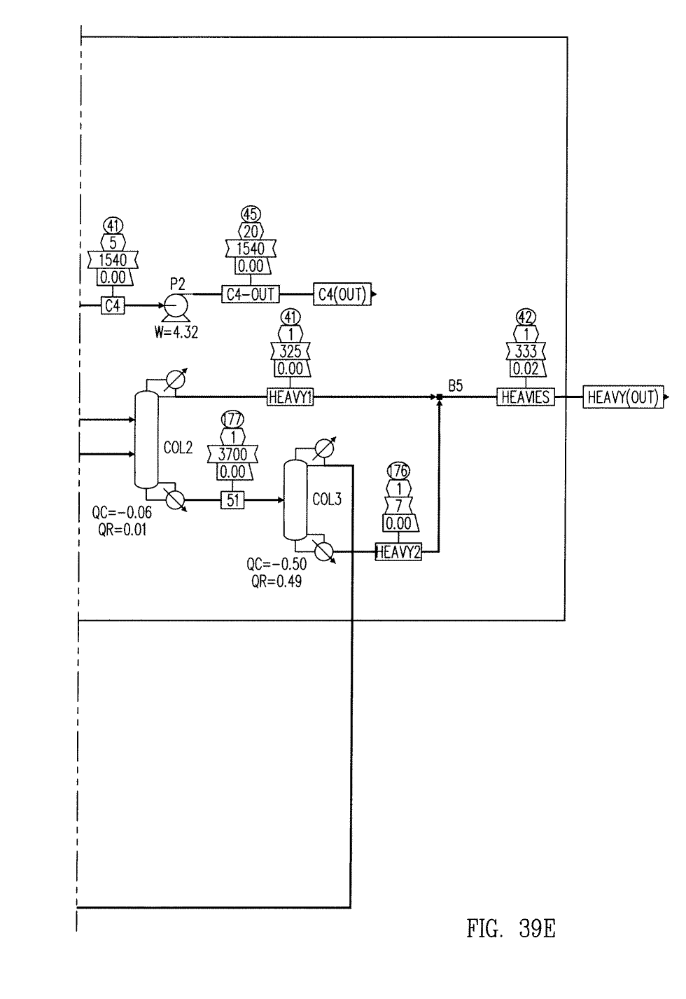

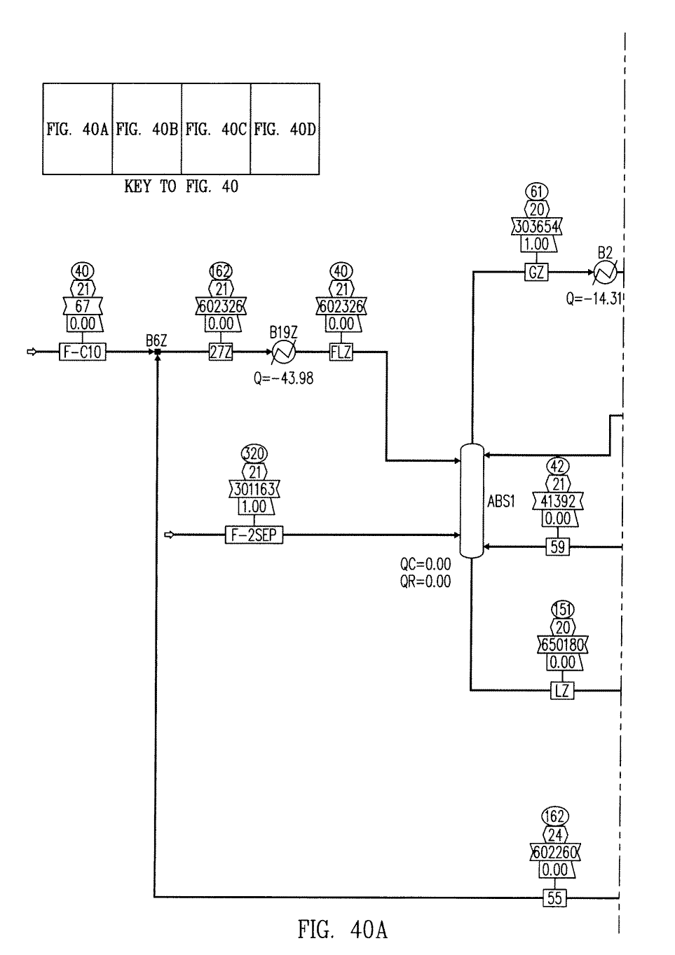

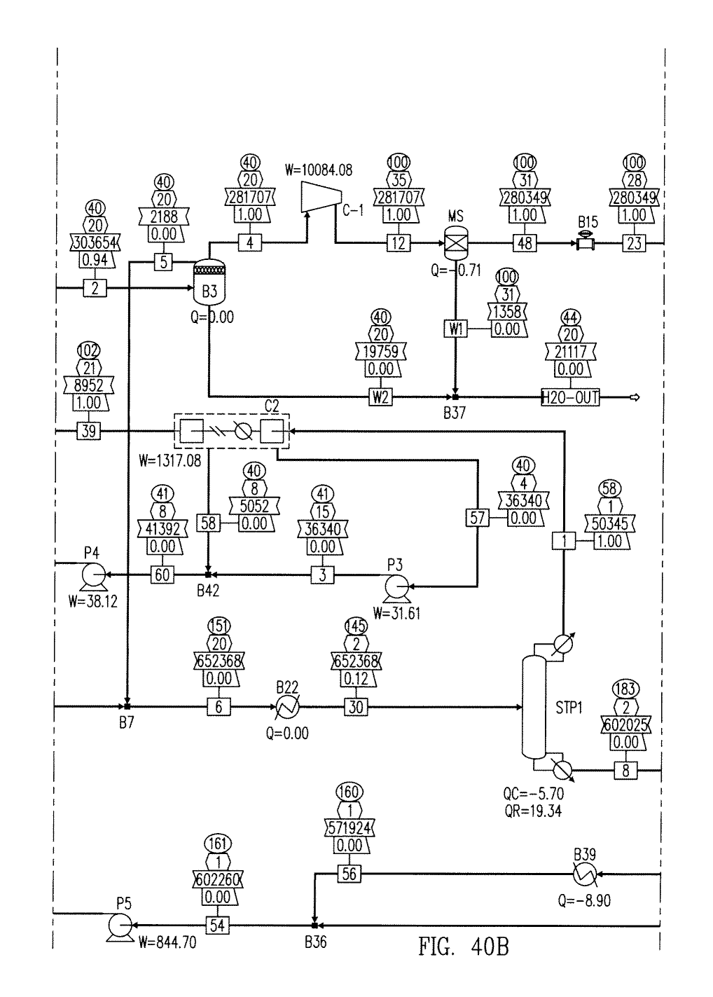

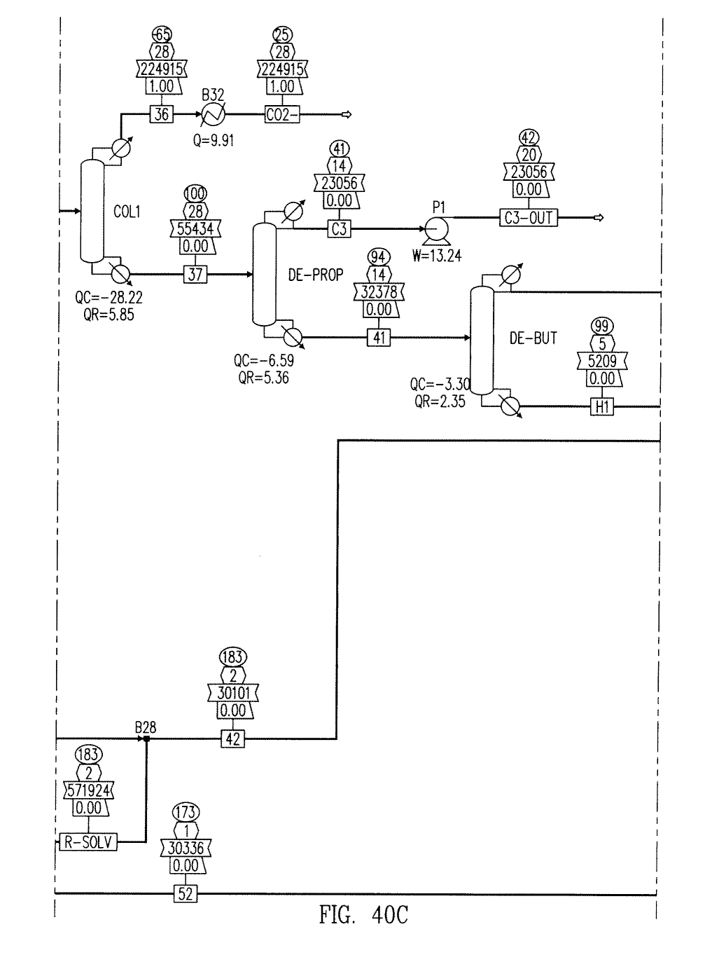

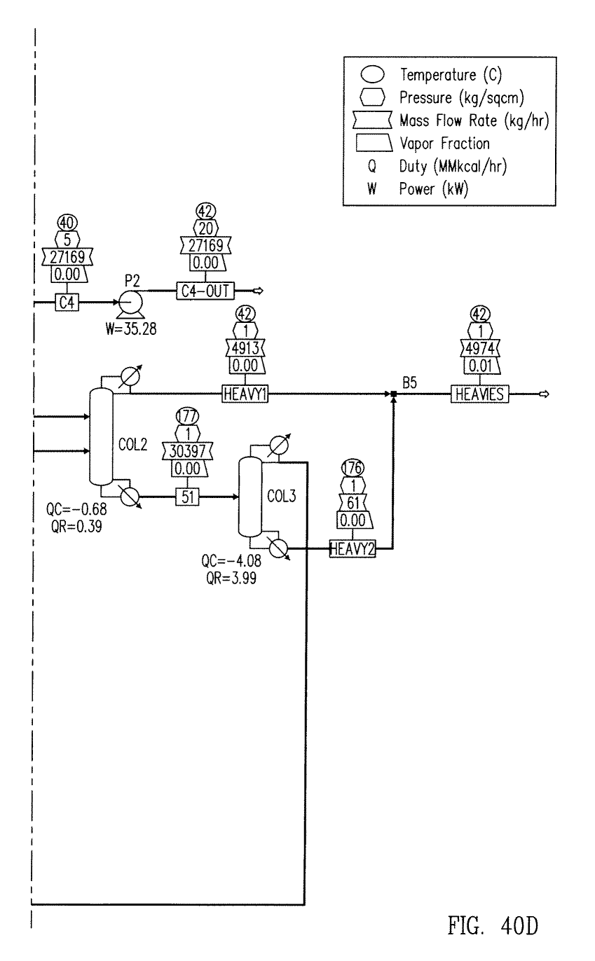

FIGS. 39 and 40 show flow diagrams from the simulation graphics of embodiment as described above. The difference between the two embodiments are mainly the compressor placement. In FIG. 39 the absorption is at high pressure so there is no need for a charge compressor to the hydrocarbon fractionation section.

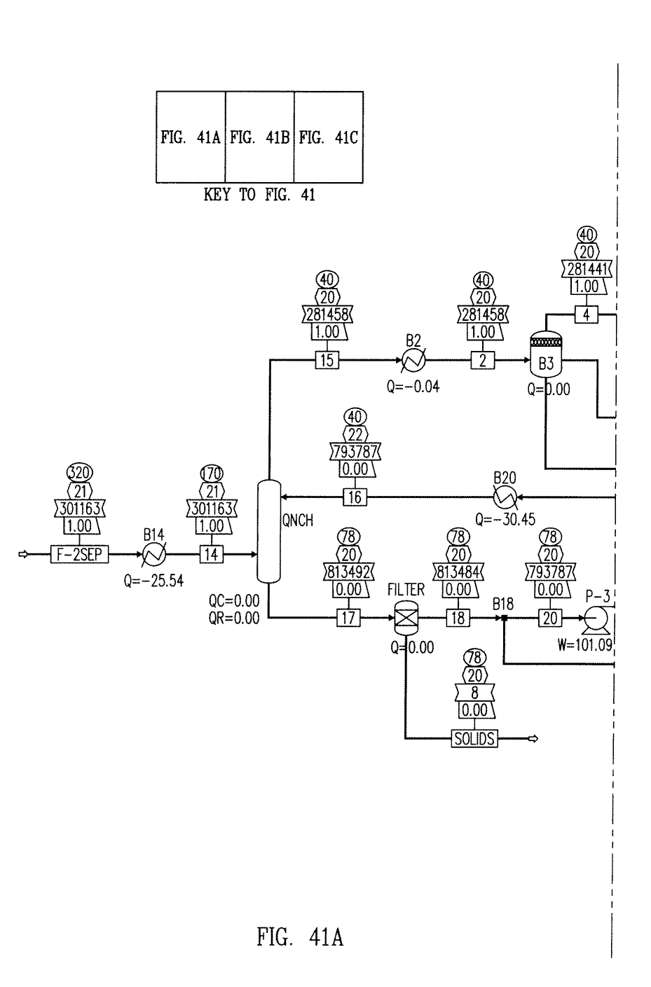

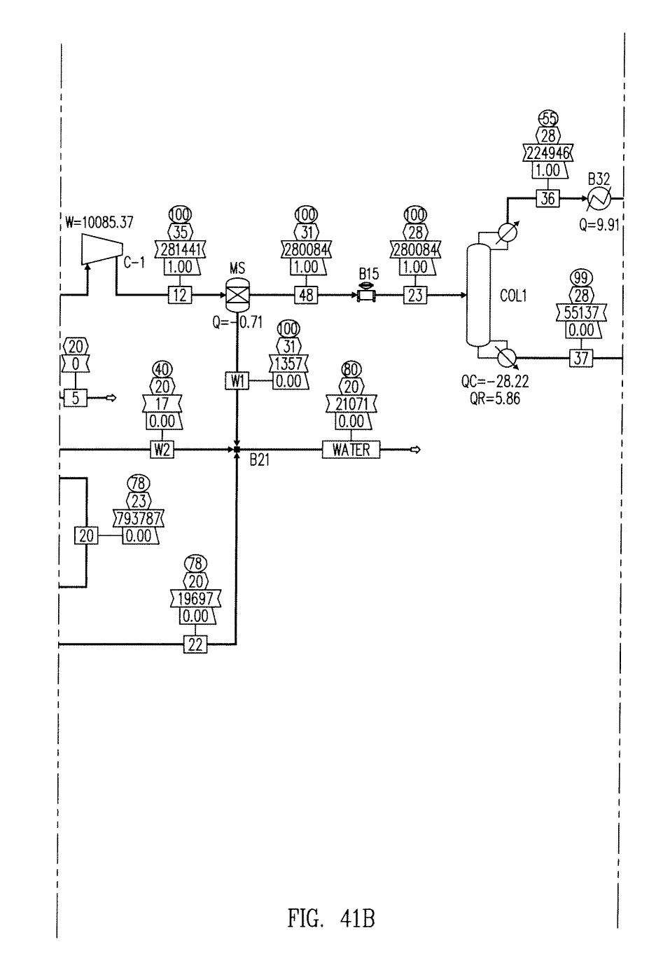

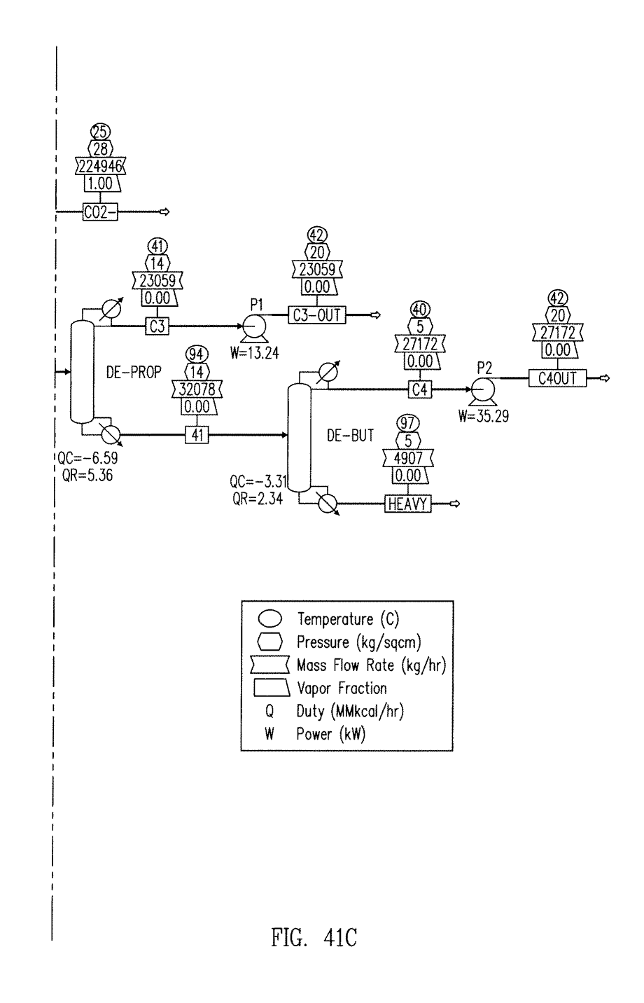

FIG. 40 illustrates another alternative embodiment. In this case, water is used as a coolant and removal method for heavy compounds. However if water is used, multiple phases or even solid formation may be present, especially if hexamethyl benzene is formed. In that case solids removal may also be needed in conjunction with the quench water washing step. Water quench may be employed as shown in FIG. 41.

Thus, as shown a variety of embodiments are possible. The present invention is not to be limited in scope by the specific embodiments disclosed in the examples which are intended as illustrations of a few aspects of the invention and any embodiments which are functionally equivalent are within the scope of this invention. Indeed, various modifications of the invention in addition to those shown and described herein will become apparent to those skilled in the art and are intended to fall within the appended claims.

We claim:

1. A method for producing liquefied petroleum gas (LPG), comprising the steps of:
reacting carbon monoxide and hydrogen in the presence of a catalyst in an LPG reactor, wherein carbon dioxide is recycled to a reformer unit and produces a feed stream to the LPG reactor, and wherein the ratio of H₂ to CO in the LPG reactor is greater than the ratio of H₂ to CO in the reformer.

2. The method of claim 1 wherein the feed stream to the LPG reactor has a ratio of H₂ to CO of up to approximately 2.0.

3. The method of claim 1 wherein the reaction is carried out at a pressure of 2.2 MPa or lower.

4. The method of claim 1 wherein the reaction is carried out at a temperature of 320° C. or lower.

5. The method of claim 1 wherein the reaction is carried out at a pressure of 6 MPa or lower.

6. The method of claim 1 wherein the reaction is carried out at a temperature in the range of 260° C. to 360° C.

7. A method for producing liquefied petroleum gas (LPG), comprising the steps of:
producing synthesis gas in a reformer unit from a carbon containing material and oxygen; and
reacting the synthesis gas in the presence of a catalyst in an LPG reactor, wherein a ratio of H₂ to CO in the LPG reactor is greater than the ratio of H₂ to CO in the reformer.

8. The method of claim 7 wherein the ratio of H₂ to CO in the LPG reactor is in the range of up to approximately 2.0.

9. The method of claim 7 wherein the reaction is carried out at a pressure of 2.2 MPa or lower.

10. The method of claim 7 wherein the reaction is carried out at a temperature of 320° C. or lower.

11. The method of claim 7 wherein the reaction is carried out at a pressure of 6 MPa or lower.

12. The method of claim 7 wherein the reaction is carried out at a temperature in the range of 260° C. to 360° C.

13. The method of claim 7 wherein the step of producing synthesis gas is carried out without steam.

14. The method of claim 7 further comprising: separating LPG in a cryogenic separation system.

15. The method of claim 7 further comprising: recycling carbon oxides to the reformer unit to selectively control the H₂ to CO ratio.

16. The method of claim 7 further comprising: recycling hydrogen to the feed stream of the LPG reactor to selectively control the H₂ to CO ratio.

17. A system for producing liquefied petroleum gas (LPG), comprising:
a reformer unit having a first ratio of H₂ to CO;
an LPG reactor having a second H₂ to CO; and
a separator system,
wherein the second ratio of H₂ to CO is greater than the first ratio of H₂ to CO.

18. The system of claim 17 wherein the separator system is comprised of a cryogenic separations system.

19. The system of claim 17 wherein the reformer unit is configured to operate without steam.
